(19) United States
(12) Patent Application Publication
(10) Pub. No.: US 2023/0078230 A1
(43) Pub. Date: Mar. 16, 2023

(54) METHODS FOR THE PRODUCTION OF COMMITTED CARDIAC PROGENITOR CELLS

(71) Applicant: FUJIFILM Cellular Dynamics, Inc., Madison, WI (US)

(72) Inventors: Steven KATTMAN, Madison, WI (US); Chad KOONCE, Madison, WI (US); Meghan BOYER, Madison, WI (US); Kristin STACK, Madison, WI (US); Ellen HEBRON, Madison, WI (US)

(73) Assignee: FUJIFILM Cellular Dynamics, Inc., Madison, WI (US)

(21) Appl. No.: 17/931,669

(22) Filed: Sep. 13, 2022

Related U.S. Application Data
(60) Provisional application No. 63/243,606, filed on Sep. 13, 2021.

(51) Int. Cl. C12N 5/0735

(52) U.S. Cl. CPC C12N 5/0606; C12N 2501/155; C12N 2506/02; C12N 2501/415; C12N 2501/15; C12N 2506/45

(57) ABSTRACT

Provided herein are methods for the differentiation of pluripotent stem cells to committed cardiac progenitor cells. Further provided herein are methods for the use of the committed cardiac progenitor cells in the treatment of cardiac disorders.

### PRIORITY CLAIM

This application claims benefit of priority to U.S. Provisional Application Ser. No. 63/243,606 filed Sep. 13, 2021, the entire contents of which are hereby incorporated by reference.

### BACKGROUND

### 1. Field

The present invention relates generally to the field of molecular biology. More particularly, it concerns the differentiation of pluripotent stem cells to committed cardiac progenitor cells.

### 2. Description of Related Art

Cardiac progenitor cells (CPCs) have the ability to differentiate to mature cardiomyocytes. These CPCs represent the latest stages of commitment to cardiomyocytes. Thus, these cells are attractive targets in drug development applications for regenerative medicine, such as for the treatment of myocardial infarction and congestive heart failure.

Current methods for producing cardiomyocytes from pluripotent stem cells require culture for a long period of time for stable contraction of cardiomyocytes. Thus, there is a need for improved methods of producing committed cardiac progenitor cells from pluripotent stem cells in a more efficient manner which requires less time in culture.

### SUMMARY

In certain embodiment, the present disclosure provides an in vitro method for producing human pluripotent stem cell (PSC)-derived committed cardiac progenitor cells comprising: (a) culturing PSCs in the presence of a Wnt agonist to initiate differentiation and a survival agent to form cell aggregates; (b) further culturing the cell aggregates in the presence of a Wnt agonist for a period of time sufficient to produce a population of mesoderm cells; and (c) differentiating the population of mesoderm cells in the presence of a Wnt inhibitor to promote cardiac specification, thereby producing a population of committed cardiac progenitor cells.

In some aspects, the PSCs are induced pluripotent stem cells (iPSCs) or embryonic stem cells (ESCs). In certain aspects, the PSCs were cultured on a surface coated by extracellular matrix prior to step (a). In some aspects, the extracellular matrix comprises vitronectin, collagen, laminin, Matrigel™, and/or fibronectin.

In certain aspects, the survival agent is a Rho-associated kinase (ROCK) inhibitor or myosin II inhibitor. For example, the ROCK inhibitor is H1152 or Y-27632. In specific aspects, the myosin II inhibitor is blebbistatin.

In some aspects, the method comprises culturing cells in suspension culture. In specific aspects, the suspension culture is performed in one or more bioreactors, such as vertical wheel bioreactors.

In certain aspects, the Wnt agonist of step (a) is CHIR 99021, SB216763, CHIR 98014, TWS119, Tideglusib, SB415286, BIO, AZD2858, AZD1080, AR-A014418, TDZD-8, LY2090314, or IM-12. In particular aspects, Wnt agonist is CHIR 99021. In specific aspects, the CHIR 99021 is present in the culture at a concentration of about 1 μM to 10 μM, such as about 1, 2, 3, 4, 5, 6, 7, 8, 9, or 10 μM, particularly about 2 μM.

In some aspects, step (a) is 1-2 days, such as about 22, 23, 24, 25, or 26 hours, particularly about 24 hours. In specific aspects, the culture of step (b) does not comprise or has essentially no insulin.

In certain aspects, the Wnt signaling agonist of step (b) is CHIR 99021, SB216763, CHIR 98014, TWS119, Tideglusib, SB415286, BIO, AZD2858, AZD1080, AR-A014418, TDZD-8, LY2090314, or IM-12. In particular aspects, the Wnt signaling agonist of step (b) is CHIR 99021. In some aspects, the CHIR 99021 is present in the culture at a concentration of 1 μM to 10 μM, such as 1, 2, 3, 4, 5, 6, 7, 8, 9, or 10 μM, particularly about 4 to 5 μM, such as about 4.4 μM.

In some aspects, the culture of step (b) further comprises an Activin/Nodal agonist and/or BMP. In some aspects, the Activin/Nodal agonist is activin A or Nodal. In certain aspects, step (b) is performed for 1 to 5 days, such as about 1, 2, 3, 4, or 5 days, particular about 1 or 2 days.

In some aspects, the mesoderm cells express KDR, PDGFRα, CXCR4, and/or CD56. In particular aspects, at least 5% (e.g., at least 6, 7, 8, 9, 10, 15, 20, 25, 30, 35, 40, 45, 50, or 55%) of the population of mesoderm cells express CD56 prior to or during step (c). In some aspects, at least 40% (e.g., at least 45, 50, 55, 60, 65, 70, or 75%) of the population of mesoderm cells express KDR and PDGFRα prior to or during step (c). In particular aspects, the cells express KDR after step (c) is initiated. In certain aspects, the population of mesoderm cells are positive for CXCR4 and CD56 prior to step (c). In some aspects, at least 30% (e.g., at least 31, 32, 33, 34, 35, 36, 37, 38, 39, 40, 45, 50, 55, 60, 65, 70, 75, or 80%) positive of the population of mesoderm cells are positive for CXCR4 and less than 60% (e.g., less than 59, 58, 57, 56, 55, 54, 53, 52, 51, 50, 45, 40, or 30%) of the population of mesoderm cells are positive for CD56 prior to step (c).

In some aspects, at least 20% (e.g., at least 21, 22, 23, 24, 25, 26, 27, 28, 29, 30, 35, 40, 45, 50, 55, 60, 65, 70, 75, or 80%) of the cells of the population of mesoderm cells are positive for CXCR4 and less than 60% (e.g., less than 59, 58, 57, 56, 55, 54, 53, 52, 51, 50, 45, 40, or 30%) of the population of mesoderm cells are positive for CD56 prior to step (c). In certain aspects, step (c) comprises adding a Wnt inhibitor when at least 20% (e.g., at least 21, 22, 23, 24, 25, 26, 27, 28, 29, 30, 35, 40, 45, 50, 55, 60, 65, 70, 75, or 80%) positive of the population of mesoderm cells are positive for CXCR4 and less than 60% (e.g., less than 59, 58, 57, 56, 55, 54, 53, 52, 51, 50, 45, 40, or 30%) of the population of mesoderm cells are positive for CD56.

In certain aspects, the Wnt inhibitor of step (c) is XAV939, IWR1, IWR2, IWR3, IWR4, ICG-001, IWR-1-endo, Wnt-059, LGK-974, LF3, CP21R7, NCB-0846, PNU-74654, or KYA179K. In particular aspects, the Wnt inhibitor is XAV939. In specific aspects, the XAV939 is present in the culture at a concentration of 5 μM to 10 μM, such as 5, 6, 7, 8, 9, or 10 μM. In some aspects, the culture of step (c) further comprises a TGFβ inhibitor, such as SB431542, LDN-193189, LY2157299, LY2109761, SB525334, SIS HCl, SB505124, GW788388, or LY364947. In particular aspects, the TGFβ inhibitor is SB431542. In specific aspects, the SB431542 is present in the culture at a concentration of 1 μM to 5 μM, such as 1, 2, 3, 4, or 5 μM. In some aspects, the culture of step (c) comprises insulin. In some aspects, the culture of step (c) further comprises a BMP inhibitor or AMPK inhibitor. In certain aspects, the BMP inhibitor is dorsomorphin, LDN193189, DMH1, DMH2, or ML 347. In some aspects, step (c) is for 1-6 days, such as 1, 2, 3, 4, 5, or 6 days, such as 1-3 days, particularly about 2 days.

In particular aspects, the method is serum free. In some aspects, the culture is performed in defined media. In some aspects, the method does not comprise performing drug resistance selection. In certain aspects, the committed cardiac progenitor cells do not express a transgene.

In some aspects, the method produces at least 1×10⁷ to 1×10¹⁰ committed cardiac progenitor cells.

In certain aspects, less than 20% (e.g., 19, 18, 17, 16, 15, 14, 13, 12, 10 or 5%) of the population of committed cardiac progenitor cells express EpCAM. In certain aspects, less than 10% (e.g., less than 9, 8, 7, 6, or 5%) of the cells of the population of committed cardiac progenitor cells express EpCAM. In some aspects, less than 20% (e.g., 19, 18, 17, 16, 15, 14, 13, 12, 10 or 5%) of the population of committed cardiac progenitor cells are positive for KDR, CXCR4, and/or SAA. In some aspects, at least 80% (e.g., 81, 82, 83, 84, 85, 85, 87, 88, 89, 90, 91, 92, 93, 94, 95, 96, 97, 98, or 99%) of the population of committed cardiac progenitor cells are positive for PDGFRα and CD56. In some aspects, less than 20% (e.g., 19, 18, 17, 16, 15, 14, 13, 12, 10 or 5%) of the population of committed cardiac progenitor cells are positive for EpCAM and SAA.

In particular aspects, the method is good manufacturing practice (GMP)-compliant. In some aspects, the method further comprises cryopreserving the population of committed cardiac progenitor cells, such as when the population of committed cardiac progenitor cells is at least 70% (e.g., 71, 72, 73, 74, 75, 75, 77, 78, 79, 80, 81, 82, 83, 84, 85, 86, 87, 88, 89, 90, 91, 92, 93, 94, 95, 96, 97, 98, or 99%) positive for PDGFRα, less than 40% (e.g., 39, 38, 37, 36, 35, 34, 33, 32, 30, 29, 28, 27, 26, 25, 24, 23, 22, 21, 20, 19, 18, 17, 16, 15, 14, 13, 12, 10 or 5%) positive for KDR, less than 20% (e.g., 19, 18, 17, 16, 15, 14, 13, 12, 10 or 5%) positive for EpCAM, and less than 20% (e.g., 19, 18, 17, 16, 15, 14, 13, 12, 10 or 5%) for SAA

In additional aspects, the method further comprises maturing the population of committed cardiac progenitor cells to produce cardiomyocytes. In some aspects, the population of committed cardiac progenitor cells are cultured in a monolayer. In certain aspects, the population of committed cardiac progenitor cells are cultured on a surface coated by extracellular matrix. In some aspects, the extracellular matrix comprises vitronectin, collagen, laminin, Matrigel™, and/or fibronectin. In specific aspects, the extracellular matrix comprises vitronectin.

In some aspects, the cardiomyocytes express CTNT, MHC, MLC, CTNI, and/or sarcomeric alpha actinin. In certain aspects, at least 80% (e.g., 81, 82, 83, 84, 85, 85, 87, 88, 89, 90, 91, 92, 93, 94, 95, 96, 97, 98, or 99%) of the cells are positive for sarcomeric alpha actinin.

In certain aspects, the culture for maturation does not comprise a Wnt inhibitor or a TGFβ inhibitor. In some aspects, the culture for maturation is 2-30 days, such as 2-20 days, such as 5-10 days.

In particular aspects, the method comprises producing primed cardiac progenitor cells comprising culturing PSCs in suspension in the presence of a Wnt agonist to initiate differentiation, culturing the cells in the presence of a Wnt inhibitor when the cell population comprises less than about 60% cells positive for CD56 and at least about 20% cells positive for CXCR4 to produce a population of primer cardiac progenitor cells are at least about 70% positive for PDGFRα, less than about 40% positive for KDR, less than about 20% positive for EPCAM, and less than about 20% positive for SAA. The population of primed cardiac progenitor cells may be cryopreserved.

In some aspects, the method further comprises differentiating the population of committed cardiac progenitor cells to a population of vascular endothelial cells. In some aspects, differentiating comprises culturing the population of committed cardiac progenitor cells in the presence of fibroblast growth factor (FGF) and/or vascular endothelial growth factor (VEGF). In certain aspects, the vascular endothelial cells are positive for CD33 and CD144. In some aspects, at least 20% (e.g., 25, 30, 35, 40, 45, 50, 55, 60, 65, 70% or higher) of the cells of the population of vascular endothelial cells are positive for CD33 and CD144.

In some aspects, the method further comprises differentiating the population of committed cardiac progenitor cells to a population of smooth muscle cells. In certain aspects, differentiating comprises culturing the population of committed cardiac progenitor cells in the presence of FGF and/or VEGF. In certain aspects, the population of smooth muscle cells are at least 50% (e.g., 55%, 60%, 70%, 75%, 80%, or more) cells positive for CD140b and CD90.

Further provided herein is a population of committed cardiac progenitor cells are produced by the methods of present embodiments and aspects thereof. Also provided herein is a population of cardiomyocytes, vascular endothelial cells, or smooth muscle cells produced by the methods of the present embodiments and aspects thereof.

Another embodiment provides a population of committed cardiac progenitor cells with at least 90% (e.g., 91, 92, 93, 94, 95, 96, 97, 98, or 99%) expression of CD56, at least 80% (e.g., 81, 82, 83, 84, 85, 86, 87, 88, 89, 90, 91, 92, 93, 94, 95, 96, 97, 98, or 99%) expression of PDGFRα and less than 10% (e.g., less than 9, 8, 7, 6, 5, 4, 3, 2, or 1%) expression of CXCR4, KDR and EpCAM. In particular aspects, the committed cardiac progenitor cells are produced by the method of present embodiments and aspects thereof. In particular aspects, the population of committed cardiac progenitor cells is GMP-compliant. In some aspects, the composition is a pharmaceutical composition.

A further embodiment provides a method for the treatment of a cardiac disorder in a subject comprising administering an effective amount of committed cardiac progenitor cells of the present embodiments or aspects thereof to a subject in need thereof.

In some aspects, the committed cardiac progenitor cells are administered directly to the heart. In certain aspects, the administration is by using an intra-myocardial catheter. In some aspects, the cells are administered in a suspension comprising human albumin (e.g., FLEXBUMIN™), such as at a concentration of 1% to 10%, such as 1, 2, 3, 4, 5, 6, 7, 8, 9, or 10%, particularly about 5%.

In some aspects, the administered committed cardiac progenitor cells show engraftment, cell survival, and maturation to cardiomyocytes. In some aspects, the subject is a human. In particular aspects, the cardiac disorder is myocardial infarction, cardiomyopathy, congestive heart failure, ventricular septal defect, atrial septal defect, congenital heart defect, ventricular aneurysm, a cardiac disorder which is pediatric in origin, ventricular aneurysm, or a cardiac disorder which requires ventricular reconstruction.

A further embodiment provides a method of generating cardiac progenitor cells, comprising providing pluripotent stem cells (PSCs), culturing the PSCs in suspension in the presence of a Wnt agonist to initiate cardiac differentiation, and adding a Wnt inhibitor when the cell population is comprised of less than about 60% CD56-positive cells and more than about 30% CXCR4-positive cells to promote robust cardiac specification, thereby producing a population of cardiac progenitor cells.

In some aspects, the cardiac progenitor cells are useful for treatment of disorders characterized by insufficient cardiac function. In certain aspects, the cardiac progenitor cells are capable of differentiation to the cardiomyocyte, endothelial and vascular smooth muscle lineages in vivo. In some aspects, the cardiac specification produces a population of committed cardiac progenitor cells (CTC4). In particular aspects, the differentiation occurs in a bioreactor.

In certain aspects, the method further comprises cryopreserving the cell population once it comprises cells more than 70% positive for PDGFRα, less than 40% positive for KRD, less than 20% positive for EPCAM, and less than 20% positive for sarcomeric alpha actinin. In some aspects, the CTC4 cells may be cryopreserved. In certain aspects, the committed cardiac progenitor cells are administered directly into the heart of a subject. In some aspects, the differentiating in the presence of a Wnt inhibitor further comprises a TGFβ inhibitor. In certain aspects, the differentiating in the presence of a Wnt inhibitor further comprises a BMP inhibitor. In some aspects, the method comprises serum free media. In certain aspects, the method does not comprise performing drug resistance selection.

In some aspects, the method further comprises maturing the population of committed cardiac progenitor cells to produce cardiomyocytes. In some aspects, the culture for maturation does not comprise a Wnt inhibitor or a TGFβ inhibitor. In particular aspects, the cells can further specify to endothelial cells or smooth muscle with the addition of VEGF.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1B: (FIG. 1A) Schematic depicting exemplary protocol for differentiation of cardiomyocyte committed cardiac progenitor cells (CTC4) from induced pluripotent stem cells (iPSCs). The process includes aggregate formation of iPSCs, mesoderm induction, and the early stages of cardiac specification. (FIG. 1B) Process day descriptions of culture methods (plated vs. suspension) and developmental milestones for cardiac differentiation.

FIGS. 2A-2B: (FIG. 2A) Process day descriptions of culture methods (plated vs. suspension) and developmental milestones for cardiac differentiation. (FIG. 2B) Schematic depicting the scale of iPSC and differentiation using multi-layer CELLSTACK® vessels and PBS3 VERTICAL-WHEEL™ bioreactors. The cells can undergo large-scale cryopreservation (e.g., 300 million cells per vial), such as with Aseptic Technologies AT CLOSED-VIALS®.

FIGS. 3A-3B: (FIG. 3A) Schematic depicting exemplary protocol for differentiation of cardiomyocyte committed cardiac progenitor cells (CTC4) from induced pluripotent stem cells (iPSCs). The process includes aggregate formation of iPSCs, mesoderm induction, and the early stages of cardiac specification. (FIG. 3B) Three different PBS3 bioreactors were sampled at differentiation days 2-5 and analyzed by flow cytometry for CXCR4 and CD56. (FIG. 3C) Committed cardiac progenitor cells were harvested from three PBS3 bioreactors, pooled, and analyzed by flow cytometry for CXCR4 and CD56.

FIGS. 4A-4B: (FIG. 4A) Three different PBS3 bioreactors were sampled at differentiation days 2-5 and analyzed by flow cytometry for EPCAM. (FIG. 4B) Committed cardiac progenitor cells were harvested from three PBS3 bioreactors, pooled, and analyzed by flow cytometry for EPCAM.

FIG. 5: PBS3 bioreactor was sampled at days 4-6 and analyzed by flow cytometry for KDR and PDGFRα. Committed cardiac progenitor cells harvested on day 6 of differentiation have greatly reduced expression of KDR.

FIGS. 6A-6B: (FIG. 6A) Gene expression from multiple batches of iPSCs and committed cardiac progenitor cells were analyzed by Fluidigm for the pluripotent genes NANOG, SOX2, and POU5F1. (FIG. 6B) Gene expression from multiple batches of committed cardiac progenitor cells were analyzed by Fluidigm for the cardiac genes HAND2, GATA4, NKX2.5, PDGFRA, and TBX5.

FIGS. 7A-7E: (FIG. 7A) Schematics depicting protocol used to confirm CTC4 cells will become cardiomyocytes after being thawed and plated into vitronectin-coated vessel in RPMI+B27 medium. (FIG. 7B) Flow cytometry characterization of CTC4 cells after being thawed showing that majority of the populations are CD56^{pos}, CXCR4^{neg}, EpCAM^{neg}, KDR^{neg}, PDGFRα^{pos}, and SAA^{neg} indicating these cells are committed to become cardiomyocytes, but have not yet started expressing the cardiac marker sarcomeric alpha actinin (SAA). (FIG. 7C) Immunocytochemistry characterization of the CTC4 cells after they have been cultured for 7 days on vitronectin-coated 96-well plates with RPMI+B27 medium. The cardiac-specific transcription factor NKX2.5 is expressed along with the cardiac structural proteins sarcomeric alpha actinin (SAA), cardiac troponin I (CTNI) and cardiac troponin T (CTNT). (FIG. 7D) Flow cytometry analysis for SAA after the CTC4 cells were cultured for 7 days on vitronectin-coated vessels with RPMI+B27 medium indicating the specification to cardiomyocytes. (FIG. 7E) Contraction of the CTC4-derived cardiomyocytes after the cells were cultured for 7 days on vitronectin-coated vessels with RPMI+B27 medium.

FIGS. 8A-8C: (FIG. 8A) Schematics depicting NUDE rat myocardial infarct model. CTC4 cells suspended in 5% Flexbumin (1e7) were administered as multiple (5) intramyocardial injections into the peri-infarct area of the left ventricle three days after the left anterior descending artery (LAD) was surgically ligated. Hearts were processed and analyzed 30 days after CTC3 injections. (FIG. 8B) Tissue was processed by cutting 5 rings per heart and embedded in paraffin. From each ring of each heart 20 serial sections were cut and taken to slide. Immunohistochemistry for human ALU was performed on slides from sections 1, 5, 10 and 20 for each heart block to determine human cellular distribution and engraftment success. (FIG. 8C) Once human cells were detected, serial sections were taken for additional processing and characterization. A multiplex method using fluorescent in-situ hybridization for human Alu followed by immunohistochemistry methods for the detection of Ki67, cardiac troponin T, or CX43 was performed to characterize the engrafted human cells.

FIGS. 9A-9B: (FIG. 9A) Schematic depicting culture of iPSC-derived cardiac progenitor cells in RPMI+B27 media comprising growth factors FGF2 and/or VEGF to differentiate to vascular endothelial (CD31+CD144+) and smooth muscle cells (CD140b+CD90+). (FIG. 9B) Flow cytometry of CD31 and CD144 expression for vascular endothelial cells and CD90 and CD140b expression for smooth muscle cells.

### DESCRIPTION OF ILLUSTRATIVE EMBODIMENTS

Differentiation of pluripotent stem cells can be induced in a variety of manners, such as in attached colonies or by formation of cell aggregates, e.g., in low-attachment environment, wherein those aggregates are referred to as embryoid bodies (EBs). The molecular and cellular morphogenic signals and events within EBs mimic many aspects of the natural ontogeny of such cells in a developing embryo. In certain embodiments, the present disclosure by providing methods for producing committed cardiac progenitor cells from pluripotent stem cells (PSCs), such as induced pluripotent stem cells (iPSCs), in large quantities and a short period of time. These committed cardiac progenitor cells are primed to become cardiomyocytes without additional growth factors or small molecule signaling, but still retain endothelial differentiation potential. In some embodiments, the differentiation process provided herein is optimized to establish stable and robust contraction quickly after thaw and plating.

The differentiation process can comprise forming aggregates from PSCs, such as iPSCs, in the presence of a Wnt agonist and an agent to promote aggregate formation, such as a ROCK inhibitor. The aggregates can then be induced to form mesoderm cells in the presence of a Wnt agonist, such as CHIR 99021. In particular aspects, the mesoderm induction medium does not comprise insulin. The mesoderm induction media may further comprise an activin agonist and/or BMP. The mesoderm cells may be identified by positive expression of CXCR4, KDR, PDGFRα, and/or CD56 as well as essentially no expression of CKIT and/or EPCAM, markers of pluripotency. The mesoderm induction step may be for about 1-3 days. Next, the mesoderm cells are subjected to cardiac specification in the presence of a Wnt inhibitor, and optionally TGFβ and/or BMP inhibitors, particularly in combination with insulin. Committed cardiac progenitor cells may be produced after initiation of cardiac specification, such as after about 1-3 days. In specific aspects, the aggregates that are at the mesoderm stage can be kept in a suspension culture system to initiate cardiac specification or the mesoderm cells may be individualized and plated as a monolayer culture prior to initiation of cardiac specification. The committed cardiac progenitor cells can be manufactured with both culture system methods. The committed cardiac progenitor cells may be further cultured to produce cardiomyocytes. In particular, the differentiation process may be serum free with no drug resistant or metabolic selection used.

In the present studies, a robust and scalable cGMP iPSC-derived cardiac differentiation protocol was developed that results in 1×10⁸-3×10⁹ committed cardiac progenitor cells (CTC4 cells) per bioreactor that can be cryopreserved at 1×10⁶-300×10⁶ cells per cryopreservation container. These CTC4 cells are distinct from previously described early stage KDR⁺ cardiac progenitor cells. For example, CTC4 cells have already rapidly decreased KDR expression and do not have the same differentiation potential as the earlier developmental staged KDR⁺ cardiac progenitor cells. Instead, the CTC4 cells may be cryopreserved at a unique later developmental stage, but before the cells have become early cardiomyocytes.

In further embodiments, the present cryopreserved cardiac progenitor cells may be thawed and cultured in media, such as RPMI+B27, to further specify toward cardiomyocytes at a high purity. The present cryopreserved cardiac progenitor cells can also become cardiomyocytes after being injected into the myocardium of a subject.

In further aspects, the present committed cardiac progenitor cells may be differentiated to vascular endothelial or smooth muscle cells, such as in media comprising FGF and/or VEGF.

In addition, the present disclosure provides therapies comprising administering the CTC4 cells provided herein. The CTC4 cells may be delivered by direct injection or by trans-endocardial, intra-myocardial catheter delivery. The dose of the iPSC-derived CTC4 cells may be about 1×10⁷ to 1×10⁹ cells. The CTC4 cells of the present disclosure may be manufactured from HLA-compatible iPSC for compatibility with subjects to be treated. The current methods may be used for cGMP manufacturing, including the use of all described materials and culture formats. Thus, the present disclosure provides a robust, reproducible, and relevant source of cells, such as to advance drug development and cardiac regenerative medicine. The CTC4 cells may also be used to identify and help avoid drug-mediated cardiac developmental toxicity problems.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects. In some aspects, the term can mean, in general, within a standard deviation of the stated value as determined using a standard analytical technique for measuring the stated value. The term can also be used by referring to plus or minus 5% of the stated value, such as for the percentage of cells in a population positive or negative for a certain marker.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs or iPS cells”.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPScs or iPS cells)” are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

“Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation and transdifferentiation.

“Reprogramming” is a process that confers on a cell a measurably increased capacity to form progeny of at least one new cell type, either in culture or in vivo, then it would have under the same conditions without reprogramming. More specifically, reprogramming is a process that confers on a somatic cell a pluripotent potential. This means that after sufficient proliferation, a measurable proportion of progeny having phenotypic characteristics of the new cell type if essentially no such progeny could form before reprogramming; otherwise, the proportion having characteristics of the new cell type is measurably more than before reprogramming.

“Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more in order of increasing preference.

The term “forward programming” refers to the programming of a multipotent or pluripotent cell, as opposed to a differentiated somatic cell that has no pluripotency, by the provision of one or more specific lineage-determining genes or gene products to the multipotent or pluripotent cell. For example, forward programming may describe the process of programming ESCs or iPSCs to hematopoietic precursor cells or other precursor cells, or to hematopoietic cells or other differentiated somatic cells.

As used herein, the term “subject” or “subject in need thereof” refers to a mammal, preferably a human being, male or female at any age that is in need of a cell or tissue transplantation. Typically, the subject is in need of cell or tissue transplantation (also referred to herein as recipient) due to a disorder or a pathological or undesired condition, state, or syndrome, or a physical, morphological or physiological abnormality which is amenable to treatment via cell or tissue transplantation.

A “survival agent” refers to an agent which promotes and/or supports cell survival when added to cell culture media. For example, Rho-associated kinase (ROCK) inhibitors or Myosin II-specific inhibitors may be used as survival agents. In particular aspects, these survival agents promote aggregation of cells in culture.

“Rho-associated kinase inhibitors,” abbreviated as “ROCK inhibitors,” refer to any substance that inhibits or reduces the function of Rho-associated kinase or its signaling pathway in a cell, such as a small molecule, an siRNA, a miRNA, an antisense RNA, or the like. “ROCK signaling pathway,” as used herein, may include any signal processors involved in the ROCK-related signaling pathway, such as the Rho-ROCK-Myosin II signaling pathway, its upstream signaling pathway, or its downstream signaling pathway in a cell. Examples of ROCK inhibitors include, but are not limited to, a Rho-specific inhibitor, a ROCK-specific inhibitor, a MRLC (myosin regulatory light chain)-specific inhibitor, or a Myosin II-specific inhibitor.

“Committed cardiac progenitor cells (CPCs), primed CPCs or CTC4 cells” are used interchangeably herein and refer to a cell whose differentiation has been manipulated towards a cardiac lineage, although it has not yet fully differentiated into a cardiomyocyte. Thus, these CPCs are primed to become cardiomyocytes. The committed cardiac progenitor cells can be cryopreserved and when plated or injected in vivo will differentiate to >90% cardiomyocytes (e.g., SAA positive cardiomyocytes) without additional growth factor or small molecule signaling. Examples of committed cardiac progenitor cell markers include PDGFRα and CD56. In particular aspects, the committed cardiac progenitor cells do not express CXCR4, KDR, CKIT, EPCAM and/or sarcomeric alpha actinin. These committed CPCs or CTC4 cells are multipotent and can also be further differentiated to other cell lineages, such as vascular endothelial cells or smooth muscle cells, such as by culturing in the presence of growth factors.

“Cardiomyocytes” or cardiac muscle cells refer to myocytes that make up the cardiac muscle. Examples of cardiac specific markers include α-sarcomeric actinin, troponin, myosin heavy chain, or L-type calcium current.

As used herein, “administering” shall mean delivering in a manner which is affected or performed using any of the various methods and delivery systems known to those skilled in the art. Administering can be performed, for example, intravenously, orally, via implant, transmucosally, transdermally, intramuscularly, or subcutaneously. Specifically, envisioned is topical administration. “Administering” can also be performed, for example, once, a plurality of times, and/or over one or more extended periods.

“Super donors” are referred to herein as individuals that are homozygous for certain MHC class I and II genes. These homozygous individuals can serve as super donors and their cells, including tissues and other materials comprising their cells, can be transplanted in individuals that are either homozygous or heterozygous for that haplotype. The super donor can be homozygous for the HLA-A, HLA-B, HLA-C, HLA-DR, HLA-DP or HLA-DQ locus/loci alleles, respectively.

### II. PLURIPOTENT STEM CELLS

In certain embodiments of the present disclosure, there are disclosed methods and compositions for providing cardiac progenitor cells from pluripotent stem cells. The pluripotent stem cells may be stem cells including but are not limited to, induced pluripotent stem cells and embryonic stem cells.

In particular aspects, the pluripotent stem cells used herein are human embryonic stem cells (ESCs) or induced pluripotent stem cells (iPSCs) which are capable of long-term proliferation in vitro, while retaining the potential to differentiate into all cell types of the body, including the cardiac progenitor cells of the present disclosure. Thus, these cells could potentially provide an unlimited supply of patient-specific functional cardiac progenitor cells for both drug development and therapeutic uses.

### A. Embryonic Stem Cells

In certain aspects, the pluripotent stem cells are embryonic stem cells (ESCs). ES cells are derived from the inner cell mass of blastocysts and have a high in vitro differentiating capability. ES cells can be isolated by removing the outer trophectoderm layer of a developing embryo, then culturing the inner mass cells on a feeder layer of non-growing cells. The replated cells can continue to proliferate and produce new colonies of ES cells which can be removed, dissociated, replated again and allowed to grow. This process of “subculturing” undifferentiated ES cells can be repeated a number of times to produce cell lines containing undifferentiated ES cells (U.S. Pat. Nos. 5,843,780; 6,200,806; 7,029,913). ES cells have the potential to proliferate while maintaining their pluripotency. For example, ES cells are useful in research on cells and on genes which control cell differentiation. The pluripotency of ES cells combined with genetic manipulation and selection can be used for gene analysis studies in vivo via the generation of transgenic, chimeric, and knockout mice.

Methods for producing mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be produced or derived from a zygote or blastocyst-staged mammalian embryo produced by the fusion of a sperm and egg cell, nuclear transfer, pathogenesis, or the reprogramming of chromatin and subsequent incorporation of the reprogrammed chromatin into a plasma membrane to produce an embryonic cell by previously described methods (Thomson and Marshall, 1998; Reubinoff et al., 2000). In one method, human blastocysts are exposed to anti-human serum, and trophectoderm cells are lysed and removed from the inner cell mass which is cultured on a feeder layer of mouse embryonic fibroblasts. Further, clumps of cells derived from the inner cell mass are chemically or mechanically dissociated, replated, and colonies with undifferentiated morphology are selected by micropipette, dissociated, and replated. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as MATRIGEL™ or laminin in the presence of “conditioned” medium containing basic fibroblast growth factor (Xu et al., 2001).

ES cells can also be derived from other organisms including rhesus monkey and marmoset by previously described methods (Thomson and Marshall, 1998; Thomson et al., 1995; Thomson and Odorico, 2000; U.S. Pat. No. 5,843,780), as well as from established mouse and human cell lines. For example, established human ES cell lines include MAOI, MA09, ACT-4, HI, H7, H9, H13, H14 and ACT30. As a further example, mouse ES cell lines that have been established include the CGR8 cell line established from the inner cell mass of the mouse strain 129 embryos, and cultures of CGR8 cells can be grown in the presence of LIF without feeder layers.

ES stem cells can be detected by protein markers including transcription factor Oct4, alkaline phosphatase (AP), stage-specific embryonic antigen SSEA-1, stage-specific embryonic antigen SSEA-3, stage-specific embryonic antigen SSEA-4, transcription factor NANOG, tumor rejection antigen 1-60 (TRA-1-60), tumor rejection antigen 1-81 (TRA-1-81), SOX2, or REX1.

### B. Induced Pluripotent Stem Cells

In other aspects, the pluripotent stem cells used herein are induced pluripotent stem (iPS) cells, commonly abbreviated iPS cells or iPSCs. The induction of pluripotency was originally achieved in 2006 using mouse cells (Yamanaka et al. 2006) and in 2007 using human cells (Yu et al. 2007; Takahashi et al. 2007) by reprogramming of somatic cells via the introduction of transcription factors that are linked to pluripotency. The use of iPSCs circumvents most of the ethical and practical problems associated with large-scale clinical use of ES cells, and patients with iPSC-derived autologous transplants may not require lifelong immunosuppressive treatments to prevent graft rejection.

With the exception of germ cells, any cell can be used as a starting point for iPSCs. For example, cell types could be keratinocytes, fibroblasts, hematopoietic cells, mesenchymal cells, liver cells, or stomach cells. T cells may also be used as a source of somatic cells for reprogramming (U.S. Pat. No. 8,741,648; U.S. Publication No. 2015/0191697). There is no limitation on the degree of cell differentiation or the age of an animal from which cells are collected; even undifferentiated progenitor cells (including somatic stem cells) and finally differentiated mature cells can be used as sources of somatic cells in the methods disclosed herein. iPS cells can be grown under conditions that are known to differentiate human ES cells into specific cell types, and express human ES cell markers including: SSEA-1, SSEA-3, SSEA-4, TRA-1-60, and TRA-1-81.

Somatic cells can be reprogrammed to produce iPS cells using methods known to one of skill in the art. One of skill in the art can readily produce iPS cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; PCT Publication NO. WO 2007/069666 A1, U.S. Pat. Nos. 8,268,620; 8,546,140; 9,175,268; 8,741,648; U.S. Patent Application No. 2011/0104125, and U.S. Pat. No. 8,691,574, which are incorporated herein by reference. Generally, nuclear reprogramming factors are used to produce pluripotent stem cells from a somatic cell. In some embodiments, at least three, or at least four, of Klf4, c-Myc, Oct3/4, Sox2, Nanog, and Lin28 are utilized. In other embodiments, Oct3/4, Sox2, c-Myc and Klf4 are utilized or Oct3/4, Sox2, Nanog, and Lin28.

Mouse and human cDNA sequences of these nuclear reprogramming substances are available with reference to the NCBI accession numbers mentioned in WO 2007/069666 and U.S. Pat. No. 8,183,038, which are incorporated herein by reference. Methods for introducing one or more reprogramming substances, or nucleic acids encoding these reprogramming substances, are known in the art, and disclosed for example, in U.S. Pat. Nos. 8,268,620, 8,691,574, 8,741,648, 8,546,140, in published U.S. Pat. No. 8,900,871 and U.S. Pat. No. 8,071,369, which are both incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may be used with the methods disclosed herein.

In certain embodiments, undefined conditions may be used; for example, pluripotent cells may be cultured on fibroblast feeder cells or a medium that has been exposed to fibroblast feeder cells in order to maintain the stem cells in an undifferentiated state. In some embodiments, the cell is cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESRTM medium (Ludwig et al., 2006a; Ludwig et al., 2006b) or E8™/Essential 8™ medium (Chen et al., 2011).

Plasmids have been designed with a number of goals in mind, such as achieving regulated high copy number and avoiding potential causes of plasmid instability in bacteria, and providing means for plasmid selection that are compatible with use in mammalian cells, including human cells. Particular attention has been paid to the dual requirements of plasmids for use in human cells. First, they are suitable for maintenance and fermentation in E. coli, so that large amounts of DNA can be produced and purified. Second, they are safe and suitable for use in human patients and animals. The first requirement calls for high copy number plasmids that can be selected for and stably maintained relatively easily during bacterial fermentation. The second requirement calls for attention to elements such as selectable markers and other coding sequences. In some embodiments, plasmids that encode a marker are composed of: (1) a high copy number replication origin, (2) a selectable marker, such as, but not limited to, the neo gene for antibiotic selection with kanamycin, (3) transcription termination sequences, including the tyrosinase enhancer and (4) a multicloning site for incorporation of various nucleic acid cassettes; and (5) a nucleic acid sequence encoding a marker operably linked to the tyrosinase promoter. In particular aspects, the plasmids do not comprise a tyrosinase enhancer or promoter. There are numerous plasmid vectors that are known in the art for inducing a nucleic acid encoding a protein. These include, but are not limited to, the vectors disclosed in U.S. Pat. Nos. 6,103,470; 7,598,364; 7,989,425; and 6,416,998, and U.S. application Ser. No. 12/478,154 which are incorporated herein by reference.

An episomal gene delivery system can be a plasmid, an Epstein-Barr virus (EBV)-based episomal vector (U.S. Pat. No. 8,546,140), a yeast-based vector, an adenovirus-based vector, a simian virus 40 (SV40)-based episomal vector, a bovine papilloma virus (BPV)-based vector, or a lentiviral vector. A viral gene delivery system can be an RNA-based or DNA-based viral vector (PCT/JP2009/062911, PCT/JP2011/069588).

### C. Embryonic Stem Cells Derived by Somatic Cell Nuclear Transfer

Pluripotent stem cells for producing the hematopoietic precursor cells could also be prepared by means of somatic cell nuclear transfer, in which a donor nucleus is transferred into a spindle-free oocyte. Stem cells produced by nuclear transfer are genetically identical to the donor nuclei. In one method, donor fibroblast nuclei from skin fibroblasts of a rhesus macaque are introduced into the cytoplasm of spindle-free, mature metaphase II rhesus macaque ooctyes by electrofusion (Byrne et al., 2007). The fused oocytes are activated by exposure to ionomycin, then incubated until the blastocyst stage. The inner cell mass of selected blastocysts are then cultured to produce embryonic stem cell lines. The embryonic stem cell lines show normal ES cell morphology, express various ES cell markers, and differentiate into multiple cell types both in vitro and in vivo.

### D. MHC Haplotype Matching

Major Histocompatibility Complex is the main cause of immune-rejection of allogeneic organ transplants. There are three major class I MHC haplotypes (A, B, and C) and three major MHC class II haplotypes (DR, DP, and DQ). The HLA loci are highly polymorphic and are distributed over 4 Mb on chromosome 6. The ability to haplotype the HLA genes within the region is clinically important since this region is associated with autoimmune and infectious diseases and the compatibility of HLA haplotypes between donor and recipient can influence the clinical outcomes of transplantation. HLAs corresponding to MHC class I present peptides from inside the cell and HLAs corresponding to MHC class II present antigens from outside of the cell to T-lymphocytes. Incompatibility of MHC haplotypes between the graft and the host triggers an immune response against the graft and leads to its rejection. Thus, a patient can be treated with an immunosuppressant to prevent rejection. HLA-matched stem cell lines may overcome the risk of immune rejection.

Because of the importance of HLA in transplantation, the HLA loci are usually typed by serology and PCR for identifying favorable donor-recipient pairs. Serological detection of HLA class I and II antigens can be accomplished using a complement mediated lymphocytotoxicity test with purified T or B lymphocytes. This procedure is predominantly used for matching HLA-A and -B loci. Molecular-based tissue typing can often be more accurate than serologic testing. Low resolution molecular methods such as SSOP (sequence specific oligonucleotide probes) methods, in which PCR products are tested against a series of oligonucleotide probes, can be used to identify HLA antigens, and currently these methods are the most common methods used for Class II-HLA typing. High resolution techniques such as SSP (sequence specific primer) methods which utilize allele specific primers for PCR amplification can identify specific MHC alleles.

MHC compatibility between a donor and a recipient increases significantly if the donor cells are HLA homozygous, i.e. contain identical alleles for each antigen-presenting protein. Most individuals are heterozygous for MHC class I and II genes, but certain individuals are homozygous for these genes. These homozygous individuals can serve as super donors and grafts generated from their cells can be transplanted in all individuals that are either homozygous or heterozygous for that haplotype. Furthermore, if homozygous donor cells have a haplotype found in high frequency in a population, these cells may have application in transplantation therapies for a large number of individuals.

Accordingly, in some embodiments, iPSCs of the present methods can be produced from somatic cells of the subject to be treated, or another subject with the same or substantially the same HLA type as that of the patient. In one case, the major HLAs (e.g., the three major loci of HLA-A, HLA-B and HLA-DR) of the donor are identical to the major HLAs of the recipient. In some cases, the somatic cell donor may be a super donor; thus, iPSCs derived from a MHC homozygous super donor may be used to generate committed cardiac progenitor cells. Thus, the committed cardiac progenitor cells derived from a super donor may be transplanted in subjects that are either homozygous or heterozygous for that haplotype. For example, the committed cardiac progenitor cells can be homozygous at two HLA alleles such as HLA-A and HLA-B. As such, committed cardiac progenitor cells produced from super donors can be used in the methods disclosed herein, to produce committed cardiac progenitor cells that can potentially “match” a large number of potential recipients.

Accordingly, certain embodiments of the present disclosure provide a repository (e.g., a library) of HLA homozygous committed cardiac progenitor cells. The HLA haplotypes represented in a subject library can reflect the most common HLA haplotypes found in human populations, e.g., common Caucasian HLA haplotypes, common HLA haplotypes found in individuals of African ancestry, common Asian HLA haplotypes, common Hispanic HLA haplotypes, common Native American HLA haplotypes, etc. For example, a single abundant haplotype can be present in a significant proportion of a population, allowing a single HLA homozygous cell line to serve as a histocompatible donor for a significant percent of patients. A library includes one, two, three, four, five, six, seven, eight, nine, 10, 10-15, 15-20, 20-25, 25-30, or more than 30 different types of HLA homozygous cells. A subject library can include a first HLA homozygous cell homozygous for a first HLA haplotype; and at least a second HLA homozygous cell homozygous for a second HLA haplotype. A subject library can include a single cell type or can include two or more different cell types. A subject library can be catalogued, e.g., by a searchable computer database, in which information regarding the HLA haplotype, and optionally additional information such as cell surface markers, karyotype information, and the like, is stored and can be searched.

The HLA homozygous committed cardiac progenitor cells described herein can find use in a broad array of clinical applications involving transplantation of cells and/or tissues. The HLA homozygous committed cardiac progenitor cells are HLA compatible with a recipient, and therefore can be introduced into the recipient without the need for immunosuppressive therapy, or at least with reduced need for immunosuppressive therapy. A standard immunosuppressive drug regimen costs thousands of dollars per month, and can have undesirable side effects, including infections and cancers that are often life-threatening and expensive to treat. The present HLA homozygous committed cardiac progenitor cells thus overcome some of the obstacles currently limiting the use of human cells for clinical applications.

### III. DIFFERENTIATION TO COMMITTED CARDIAC PROGENITOR CELLS (CTC4)

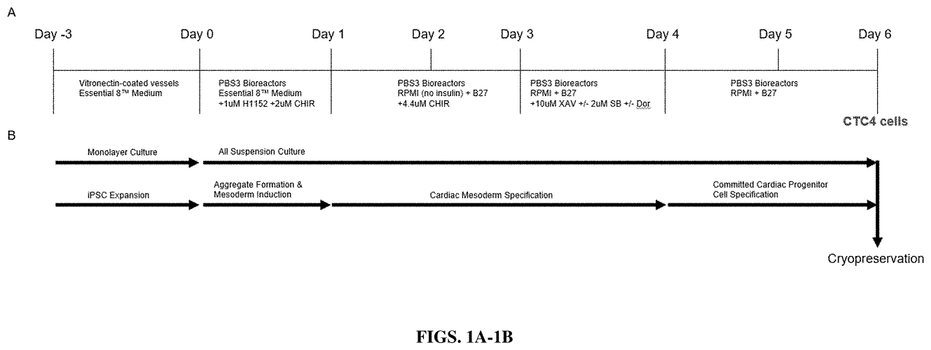

Embodiments of the present disclosure concern the differentiation of PSCs, particularly iPSCs, to cardiac progenitor cells that are committed or primed to further differentiate to cardiomyocytes. A schematic in FIG. 1A shows an exemplary 6-day differentiation process which begins with using iPSCs that have been expanded on vitronectin-coated vessels with Essential 8 medium before initiating differentiation, such as large-scale differentiation in bioreactors.

In some aspects, the present method involves the modulation of Wnt signaling in a full suspension bioreactor process. During cardiac development the signaling of Wnt in mesoderm can be rapidly modulated to drive further cardiac specification. To date, the timing of when Wnt signaling must be decreased has not been well described. In the present studies, the expression of two cell surface markers, CXCR4 and CD56, was tracked. Cardiac differentiation can be tracked by analyzing these two markers daily and decisions can be made based on the expression profiles to have robust cardiac differentiation. The initial mesoderm stage is indicated by a CXCR4⁺D56⁻ population, followed by double positive CXCR4⁺CD56⁺ cells before losing expression of CXCR4 and resulting in CXCR4⁻CD56⁺ committed cardiac progenitor cells.

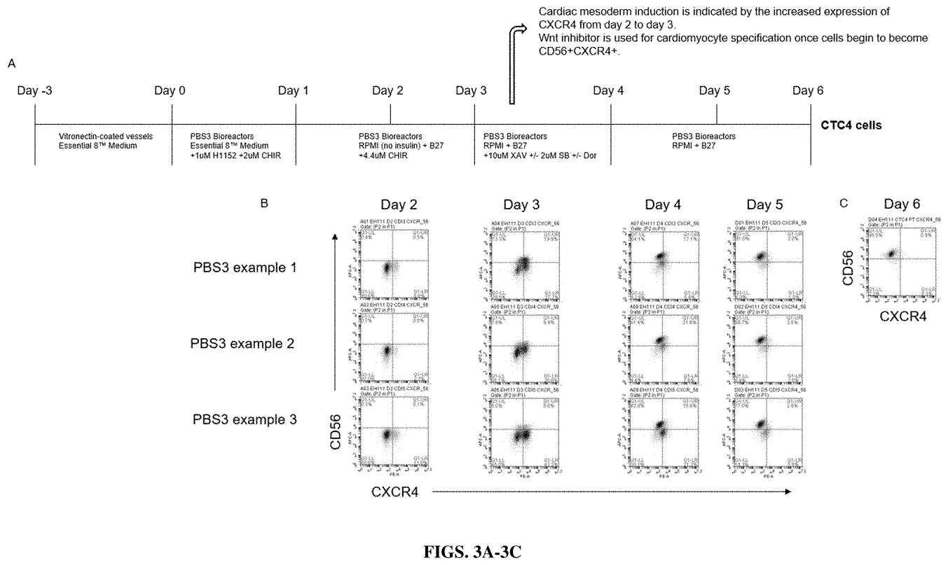

Wnt signaling may be inhibited in order to have robust cardiac specification that will drive cells to become cardiomyocytes, as shown in FIG. 3B. Preferably, the day 3 cultures are at least 30% positive for CXCR4 and less than 60% positive for CD56. If the cultures become more than 60% positive CD56 before Wnt signaling is inhibited, there may not be robust specification of cardiac cells and, thus, have a low efficiency of becoming cardiomyocytes. In addition, if the cultures have already become more than 20% CXCR4⁻CD56⁺, indicating the loss of CXCR4 expression, it is also too late to inhibit Wnt signaling for robust cardiac specification.

### A. Aggregate Formation

The pluripotent stem cells are differentiated to CTC4 cells by first inducing the formation of aggregates along with initiating differentiation with a Wnt agonist, such as CHIR 99021. Upon aggregation, differentiation is initiated and the cells begin to a limited extent recapitulate embryonic development. Though they cannot form trophectodermal tissue (which includes the placenta), cells of virtually every other type present in the organism can develop. The present disclosure may further promote cardiac differentiation following aggregate formation.

Pluripotent cells may be allowed to form embryoid bodies or aggregates as a part of the differentiation process. The formation of “embryoid bodies” (EBs), or clusters of growing cells, in order to induce differentiation generally involves in vitro aggregation of human pluripotent stem cells into EBs and allows for the spontaneous and random differentiation of human pluripotent stem cells into multiple tissue types that represent endoderm, ectoderm, and mesoderm origins.

In particular embodiments, the pluripotent stem cells are cultured in the presence of a ROCK inhibitor and a chemical agonist of the Wnt pathway, such as a GSK3 inhibitor (e.g., CHIR 99021), to stimulate the Wnt pathway. Agonists of the Wnt pathway may include CAS 853220-52-7 (2-Amino-4-(3,4-(methylenedioxy)benzylamino)-6-(3-methoxyphenyl)pyrimidine), SB216763, CHIR 98014, TWS119, Tideglusib, SB415286, BIO, AZD2858, AZD1080, AR-A014418, TDZD-8, LY2090314, or IM-12. The medium may comprise the Wnt agonist, such as CHIR 99021, at a concentration of about 1-10 μM, such as about 1, 2, 3, 4, 5, 6, 7, 8, 9, or 10 μM. In particular aspects, the medium comprises the Wnt agonist, such as CHIR 99021, at a concentration of about 4.4 μM. In particular aspects, the method comprises culturing the cells in the presence of about 2 μM of the Wnt agonist during aggregate formation, such as day 0 to day 1, and then in the presence of about 4.4 μM, such as from day 1 to day 3, for mesoderm induction.

ROCK inhibitors may be used for culturing and passaging of pluripotent stem cells and/or differentiation of the stem cells. Therefore, ROCK inhibitors could be present in any cell culture medium in which pluripotent stem cells grow, dissociate, form aggregates, or undergo differentiation, such as an adherent culture or suspension culture. Rho-specific inhibitors, such as Clostridium botulinum C3 exoenzyme, and/or Myosin II-specific inhibitors may also be used as a ROCK inhibitor in certain aspects of the present disclosure. In specific aspects, myosin II inhibitors, such as blebbistatin, can be used to induce aggregate formation.

An exemplary ROCK-specific inhibitor is Y-27632, which selectively targets ROCK1 (but also inhibits ROCK2), as well as inhibits TNF-α and IL-1β. It is cell permeable and inhibits ROCK1/ROCK2 (IC₅₀=800 nM) by competing with ATP. Other ROCK inhibitors include, e.g., H1152, Y-30141, Wf-536, HA-1077, hydroxyl-HA-1077, GSK269962A and SB-772077-B. In particular aspects, the ROCK-specific inhibitor used in the present methods is H1152. In some aspects, H1152 is present in the culture at a concentration of 50-200 μM, such as about 100 μM.

Other non-limiting examples of ROCK inhibitors include antisense nucleic acids for ROCK, RNA interference inducing nucleic acid (for example, siRNA), competitive peptides, antagonist peptides, inhibitory antibodies, antibody-ScFV fragments, dominant negative variants and expression vectors thereof. Further, since other low molecular compounds are known as ROCK inhibitors, such compounds or derivatives thereof can be also used in embodiments (for example, refer to U.S. Patent Publication Nos. 20050209261, 20050192304, 20040014755, 20040002508, 20040002507, 20030125344 and 20030087919, and International Patent Publication Nos. 2003/062227, 2003/059913, 2003/062225, 2002/076976 and 2004/039796, which are hereby incorporated by reference). In the present methods, a combination of one or two or more of the ROCK inhibitors can also be used.

According to some embodiments, the PSCs can be treated with a ROCK inhibitor in a medium. Thereby, the medium used in the methods of the present disclosure may already contain the ROCK inhibitor or alternatively, the methods of the present disclosure may involve a step of adding the ROCK inhibitor to the medium. The concentration of the ROCK inhibitor in the medium is particularly not limited as far as it can achieve the desired effects such as the improved survival rate of stem cells. Such a ROCK inhibitor, e.g., Y-27632, HA-1077, or H-1152, may be used at an effective concentration of at least or about 0.02, 0.05, 0.1, 0.2, 0.5, 1, 2, 3, 4, 5, 10, 15, 20, 25, 30, 35, 40, 45, 50, 100, 150, 200, 500 to about 1000 μM, or any range derivable therein. These amounts may refer to an amount of a ROCK inhibitor individually or in combination with one or more ROCK inhibitors.

For example, when Y-27632 is used as the ROCK inhibitor, it can be used at the concentration of about 0.01 to about 1000 μM, more specifically about 0.1 to about 100 μM, further more specifically about 1.0 to about 30 μM, and most specifically about 2.0 to 20 μM, or any range derivable therein. When Fasudil/HA1077 is used as the ROCK inhibitor, it can be used at about twofold the aforementioned Y-27632 concentration. When H1152 is used as the ROCK inhibitor, it can be used at about 1/50th of the aforementioned Y-27632 concentration.

The aggregate formation step is performed for a duration of time sufficient to induce the production of aggregates. For example, the pluripotent stem cells, such as induced pluripotent stem cells, may be contacted with the ROCK inhibitor for about 10, 15, 20, 25, 30 minutes to several hours (e.g., at least or about one hour, two hours, three hours, four hours, five hours, six hours, eight hours, 12 hours, 16 hours, 24 hours, 36 hours, 48 hours, or any range derivable therein). In particular aspects, a period of 1-3 days, such as about 1 day, is sufficient to induce the cells to form aggregates.

The density of the stem cell(s) to be treated with the ROCK inhibitor is particularly not limited as far as it is a density at which the desired effects such as the improved survival rate of stem cells can be achieved. It is, for example, about 1.0×10¹ to 1.0×10⁷ cells/ml, more particularly about 1.0×10² to 1.0×10⁷ cells/ml, further more particularly about 1.0×10³ to 1.0×10⁷ cells/ml, and most particularly about 3.0×10⁴ to 2.0×10⁶ cells/ml.

In certain embodiments, PSCs are cultured in the presence of ROCK inhibitors to improve survival at low density (dissociated into single cells or small aggregates), cloning efficiency or passaging efficiency. In certain embodiments, the PSCs are cultured in the absence of feeder cells, feeder cell extracts and/or serum. The PSCs can be cultured in the presence of a ROCK inhibitor prior to subcloning or passaging, e.g., for at least one hour before subcloning or passaging. Alternatively or additionally, the PSCs are maintained in the presence of a ROCK inhibitor during or after subcloning or passaging.

Pluripotent stem cells may be seeded into aggregate promotion medium using any method known in the art of cell culture. For example, pluripotent stem cells may be seeded as a single colony or clonal group into aggregate promotion medium, and pluripotent stem cells may also be seeded as essentially individual cells. In some embodiments, pluripotent stem cells are dissociated into essentially individual cells using mechanical or enzymatic methods known in the art. By way of non-limiting example, pluripotent stem cells may be exposed to a proteolytic enzyme which disrupts the connections between cells and the culturing surface and between the cells themselves. Enzymes which may be used to individualize pluripotent stem cells for aggregate formation and differentiation may include, but are not limited to, trypsin, in its various commercial formulations, such as TrypLE, or a mixture of enzymes such as Accutase®.

Various matrix components may be used to culture the pluripotent cells including a collagen (e.g., collagen IV), laminin, vitronectin, Matrigel™, gelatin, polylysine, thrombospondin (e.g., TSP-1, -2, -3, -4 and/or -5), fibronectin, and/or ProNectin-F™. Combinations of these matrix components may provide additional benefit for promoting cell growth and cell viability. In certain embodiments, 1, 2, 3, 4, 5, 6, or more of the above matrix components may be used to culture cells. In some aspects, the pluripotent cells are cultured on a vitronectin-coated surface.

In certain embodiments, pluripotent cells may be added or seeded as essentially individual (or dispersed) cells to a culturing medium for culture formation on a culture surface. The culturing medium into which cells are seeded may comprise Essential 8 (E8) medium, a survival factor, such as ROCK inhibitor, and a Wnt pathway agonist. In these embodiments, a culturing surface may be comprised of essentially any material which is compatible with standard aseptic cell culture methods in the art, for example, a non-adherent surface. A culturing surface may additionally comprise a matrix component (e.g., vitronectin) as described herein. In certain embodiments, a matrix component may be applied to a culturing surface before contacting the surface with cells and medium.

### B. Mesoderm Induction

Next, the pluripotent stem cell aggregates, such as iPS cell aggregates, are cultured in medium to promote mesoderm induction. The aggregates may be contacted with a Wnt agonist, and optionally a Activin/Nodal agonist and/or BMP. In particular aspects, the medium does not comprise a ROCK inhibitor or insulin. The medium may comprise a higher concentration of one or more Wnt agonists as compared to the aggregate formation step. The Wnt agonist may be the same as the Wnt agonist in the aggregate formation step or may be a different Wnt agonist. Agonists of the Wnt pathway may include CHIR 99021, IWP-1, IWP-2, IWP-3, IWP-4, CAS 853220-52-7 (2-Amino-4-(3,4-(methylenedioxy)benzylamino)-6-(3-methoxyphenyl)pyrimidine), SB216763, CHIR 98014, TWS119, Tideglusib, SB415286, BIO, AZD2858, AZD1080, AR-A014418, TDZD-8, LY2090314, or IM-12. The Wnt agonist may be CHIR 99021 and may be present at a concentration of about 1-10 μM, such as about 1, 2, 3, 4, 5, 6, 7, 8, 9, or 10 μM. In particular aspects, the Wnt agonist is CHIR 99021 and is present at a concentration of about 4-5 μM, such as about 4.1, 4.2, 4.3, 4.4, 4.5, 4.6, 4.7, 4.8, 4.9, or 5 μM, specifically about 4.4 μM.

An activin agonist is a compound which activates the Activin/Nodal signaling pathway, for example by binding to TGFβ or activin receptors. Examples of activin agonists include activin A, activin B, activin AB, TGFβl, Growth and Differentiation Factor (GDF)-3, BML-284 and Nodal. For example, an activin agonist or BMP may be used at a concentration of 0.1 ng/mL to 12 ng/mL.

The basal medium for mesoderm induction may be any medium known in the art for culturing stem cells. Exemplary medium include E8, TeSR, BME, BGJb, CMRL 1066, Glasgow MEM, Improved MEM Zinc Option, IMDM, Medium 199, Eagle MEM, αMEM, DMEM, Ham, RPMI 1640, and Fischer's media. In particular aspects, the basal medium is RPMI supplemented with B27. In specific aspects, the media does not comprise or has essentially no insulin.

The mesoderm induction step may be for a period of time sufficient to induce mesoderm markers, such as CXCR4, KDR, PDGFRα, and/or CD56, as well as loss of expression of CKIT and/or EPCAM. For example, the aggregates may be cultured in the presence of Wnt agonst, Activin/Nodal agonist, and/or BMP for about 1-5 days, such as about 1, 2, 3, 4, or 5 days. In particular aspects, the aggregates are cultured for about 2-3 days for mesoderm induction.

In particular aspects, at the early mesoderm stage, such as day 2, CXCR4 begins to be expressed. Next, a more robust expression of CXCR4 is detected along with the beginning of CD56 expression.

### C. Cardiac Specification

The mesoderm cells are then directed to cardiac specification in the presence of a Wnt inhibitor and, optionally, a TGFβ inhibitor. The culture may further comprise insulin, an activin inhibitor, and/or a BMP inhibitor. The cardiac specification may be promoted by the addition of insulin. The Wnt inhibitor may be added once the cells are less than 60% positive for CD56 and at least 20% positive for CXCR4. After Wnt inhibition, such as Day 4, the cells are at early cardiac mesoderm stage characterized by loss of CXCR4 expression with a majority of the cells expressing CD56 and a KDR+PDGRFα+ population emerging. On Day 5, the KDR+PDGFRα+ cardiac progenitor cell population is seen along with continued expression of CD56 and loss of CXCR4 expression. Then, such as on Day 6, a primed or committed cardiac progenitor population emerges as characterized by loss of KDR expression.

In particular aspects, the aggregates that are at the mesoderm stage can be kept in a suspension culture system to initiate cardiac specification or the mesoderm cells may be individualized and plated as a monolayer culture prior to initiation of cardiac specification. The CTC4 cells can be manufactured with both culture system methods. The CTC4 cells may be further cultured to produce cardiomyocytes. In particular, the differentiation process may be serum free with no drug resistant or metabolic selection used.

The Wnt inhibitor may be XAV939, ICG-001, IWR-1-endo, Wnt-059, LGK-974, LF3, CP21R7, NCB-0846, PNU-74654, IWR-1, IWR-2, IWR-3, IWR-4 or KYA179K. The Wnt inhibitor, such as XAV939, may be present at a concentration of about 1-25 mM, such as about 5, 10, or 15 mM, particularly about 10 mM.

The TGFβ inhibitor may be SB431542, LDN-193189, LY2157299, LY2109761, SB525334, SIS HCl, SB505124, GW788388, or LY364947. The TGFβ inhibitor, such as SB431542, may be present at a concentration of about 1-25 mM, such as about 5, 10, or 15 mM, particularly about 10 mM. 193189, LY2157299, LY2109761, SB525334, SIS HCl, SB505124, GW788388, or LY364947. The TGFβ inhibitor, such as SB431542, may be present at a concentration of about 1-5 μM, such as about 1, 2, or 3 μM, particularly about 2 μM.

The BMP inhibitor may be 6-[4-[2-(1-Piperidinyl)ethoxy]phenyl]-3-(4-pyridinyl)-pyrazolo[1,5-a]pyrimidine dihydrochloride (Dorsomorphin), 4-(6-(4-(piperazin-1-yl)phenyl)pyrazolo[1,5-a]pyrimidin-3-yl)quinoline hydrochloride (LDN193189), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347). The BMP inhibitor, such as dorsomorhpin, may be present at a concentration of about 0.1 μM to 5 μM, such as about 1, 2, or 3 μM, particularly about 2 μM.

CTC4 cells may be produced from mesoderm about 1 to 4 days after initiation of cardiac specification. The cardiac specification may be performed for about 1-4 days, such as about 2 or 3 days. The CTC4 cells may then be cryopreserved or differentiated to cardiomyocytes in appropriate medium, such as RPMI with B27 supplement. In particular aspects, the committed cardiac progenitor cells may be isolated or cryopreserved once the cell population is at least 70% positive for PDGFRα, less than 40% positive for KDR, less than 20% positive for EPCAM, and less than 20% positive for SAA.

The aggregates of committed cardiac progenitor cells may be dissociated and cryopreserved. Aggregate dissociation can be performed using any known procedures. These procedures include treatments with a chelating agent (such as EDTA), an enzyme (such as trypsin, collagenase), or the like, and operations such as mechanical dissociation (such as pipetting). The cells may be cultured on a matrix as described above, such as a vitronectin-coated surface.

### D. CTC4 Differentiation to Cardiomyocytes

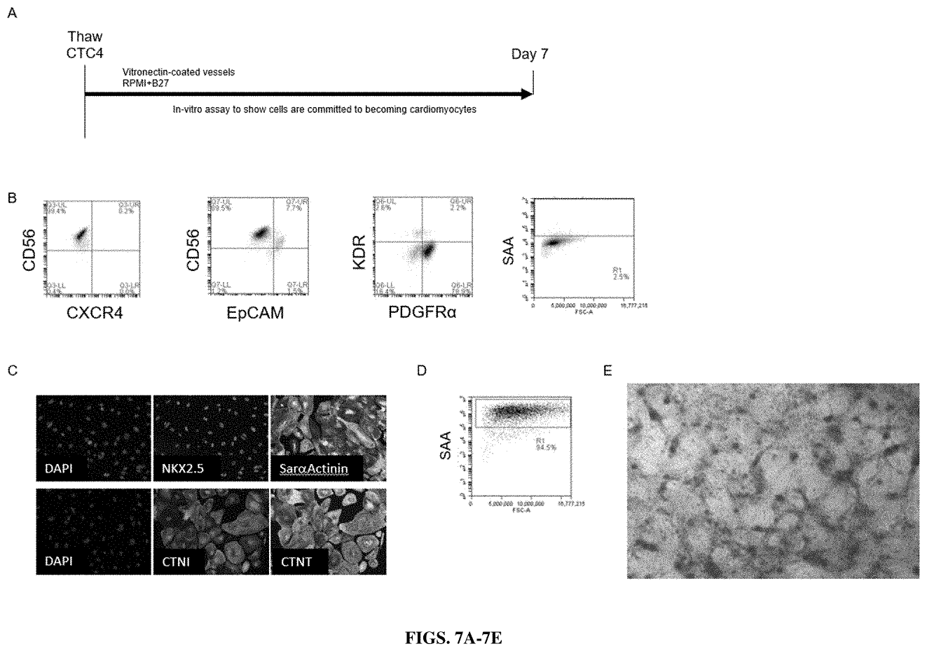

As described in FIG. 7A, the CTC4 cells may be further matured or differentiated to cardiomyocytes. In particular, the CTC4 cells may be matured to subpopulations of cardiomyocytes, such as atrial, ventricular, and pacemaker cells by differentiation conditions known in the art. When plated into various size vessels, the CTC4 cells can differentiate to a high purity and contracting monolayer of cardiomyocytes (FIGS. 7C-E).

To promote the cardiomyocyte phenotype, the cells can be cultured with factors and factor combinations that enhance proliferation or survival of cardiomyocyte type cells, or inhibit the growth of other cell types. The effect may be due to a direct effect on the cell itself, or due to an effect on another cell type, which in turn enhances cardiomyocyte formation. For example, factors that induce the formation of hypoblast or epiblast equivalent cells, or cause these cells to produce their own cardiac promoting elements, all come within the rubric of cardiotropic factors or differentiation factors for cardiomyocyte differentiation.

For example, induction medium for cardiac differentiation may include, but is not limited to, precardiac explants, precardiac mesoderm conditioned medium, mesoderm secreted growth factors such as HGF. In a particular aspect, the differentiation factors may be growth factors that are involved in cell development. The differentiation factors may include, but not be limited to, one or more of modulators of signaling pathways of bone morphogenetic protein, ActivinA/Nodal, vascular endothelial growth factor (VEGF), dickkopf homolog 1 (DKK1), basic fibroblast growth factor (bFGF), insulin growth factor (IGF), and/or epidermal growth factor (EGF).

The CTC4 cells may be cultured in medium to promote maturation to cardiomyocytes. An exemplary maturation media may comprise RPMI with B27 supplement. In some aspects, the term “maturation media” refers to medium used to further differentiate the cells to produce a cell population that is more than 70% positive for PDGFRα, less than 40% positive for KRD, less than 20% positive for EPCAM, and less than 20% positive for sarcomeric alpha actinin. For example, the cells may differentiate into cardiomyocytes or endothelial cells.

In one method, the CTC4 cells are matured to cardiomyocytes in medium supplemented with a Wnt inhibitor and a TGFβ inhibitor as described above. For example, the medium may be Williams E Medium with cell maintenance cocktail B (i.e., penicillin/streptomycin, insulin, trasnferrin, selenous acid, BSA, linoleic acid, GlutaMAX, and HEPES), a Wnt inhibitor (e.g., XAV939), and a TGFβ inhibitor (e.g., SB431542). Alternatively, to the TGFβ inhibitor or in addition to the TGFβ inhibitor, the CTC4 cells may be contacted with an Activin inhibitor and/or a BMP inhibitor. The cells may be cultured in monolayer, such as on an extracellular matrix coating (e.g., vitronectin).

### E. Cell Culture Conditions

The culturing conditions according to the present disclosure will be appropriately defined depending on the medium and stem cells used. The medium according to the present disclosure can be prepared using a medium to be used for culturing animal cells as its basal medium. As the basal medium, any of E8, TeSR, BME, BGJb, CMRL 1066, Glasgow MEM, Improved MEM Zinc Option, IMDM, Medium 199, Eagle MEM, αMEM, DMEM, Ham, RPMI 1640, and Fischer's media, as well as any combinations thereof can be used, but the medium is not particularly limited thereto as far as it can be used for culturing animal cells.

In particular aspects, the medium according to the present disclosure is a serum-free medium. The serum-free medium refers to media with no unprocessed or unpurified serum and accordingly, can include media with purified blood-derived components or animal tissue-derived components (such as growth factors). The medium according to the present disclosure may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example. Alternatively, any commercially available materials can be used for more convenience. The commercially available materials include knockout Serum Replacement (KSR), Chemically-defined Lipid concentrated (Gibco), and Glutamax (Gibco).

The medium of the present disclosure can also contain fatty acids or lipids, amino acids (such as non-essential amino acids), vitamin(s), growth factors, cytokines, antioxidant substances, 2-mercaptoethanol, pyruvic acid, buffering agents, and inorganic salts. The concentration of 2-mercaptoethanol can be, for example, about 0.05 to 1.0 mM, and particularly about 0.1 to 0.5 mM, but the concentration is particularly not limited thereto as long as it is appropriate for culturing the stem cell(s).

A culture vessel used for culturing the stem cell(s) can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, roller bottle, and bioreactors, such as PBS500 and/or PBS3, as long as it is capable of culturing the stem cells therein. The stem cells may be culture in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, 2000 ml, or any range derivable therein, depending on the needs of the culture. In a certain embodiment, the culture vessel may be a bioreactor, which may refer to any device or system that supports a biologically active environment. The bioreactors may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, and fibronectin and mixtures thereof for example Matrigel™, and lysed cell membrane preparations (Klimanskaya et al., 2005).

Other culturing conditions can be appropriately defined. For example, the culturing temperature can be about 30 to 40° C., for example, at least or about 31, 32, 33, 34, 35, 36, 37, 38, 39° C. but particularly not limited to them. The CO₂ concentration can be about 1 to 10%, for example, about 2 to 5%, or any range derivable therein. The oxygen tension can be at least or about 1, 5, 8, 10, 20%, or any range derivable therein.

The methods of the present disclosure can be also used for a suspension culture of stem cells, including suspension culture on carriers (Fernandes et al., 2007) or gel/biopolymer encapsulation (United States Patent 20070116680). The term suspension culture of the stem cells means that the stem cells are cultured under non-adherent condition with respect to the culture vessel or feeder cells (if used) in a medium. The suspension culture of stem cells includes a dissociation culture of stem cells and an aggregate suspension culture of stem cells. The term dissociation culture of stem cells means that suspended stem cells is cultured, and the dissociation culture of stem cells include those of single stem cell or those of small cell aggregates composed of a plurality of stem cells (for example, about 2 to 400 cells). When the aforementioned dissociation culture is continued, the cultured, dissociated cells form a larger aggregate of stem cells, and thereafter an aggregate suspension culture can be performed. The aggregate suspension culture includes an embryoid culture method (see Keller et al., 1995), and a SFEB method (Watanabe et al., 2005); International Publication No. 2005/123902). The methods of the present disclosure can significantly improve the survival rate and/or differentiation efficiency of stem cells in a suspension culture.

Bioreactors can be grouped according to general categories including: static bioreactors, stirred flask bioreactors, rotating wall vessel bioreactors, hollow fiber bioreactors and direct perfusion bioreactors. Within the bioreactors, cells can be free, or immobilized, seeded on porous 3-dimensional scaffolds (hydrogel). In certain aspects, the bioreactor is a suspension bioreactor for efficient mixing with homogeneous particle suspension and low shear stress.

### F. GMP Manufacturing Process

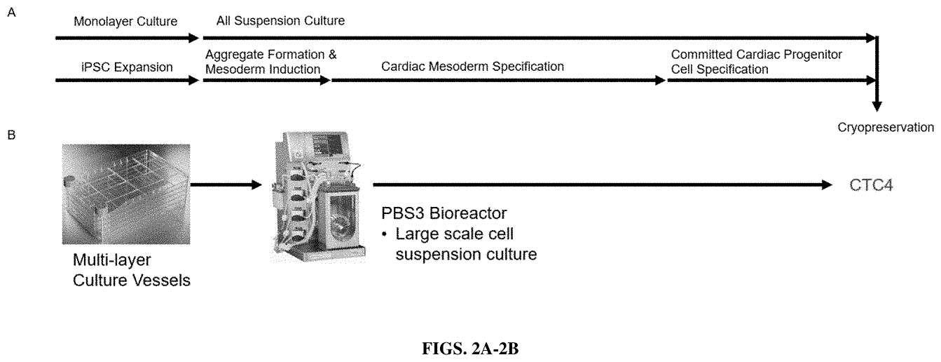

The methods disclosed utilized herein may use all GMP compatible materials and be scaled to multiple (e.g., 3 L) bioreactor manufacturing batches to yield the purity and cell numbers needed for cardiac cell therapy development. As shown in FIG. 2B, the scale of iPSC expansion in multilayer culture vessels yields enough iPSCs needed to seed multiple 3 L bioreactors. The CTC4 cryopreservation step during manufacturing may be scaled to freeze up to 300×10⁶ CTC4 cells per vial (FIG. 2C) which can reduce vial thawing and handling during preclinical development in large animal models or future clinical studies.

### G. Characterization of Committed Cardiac Progenitor Cells (CTC4 cells)

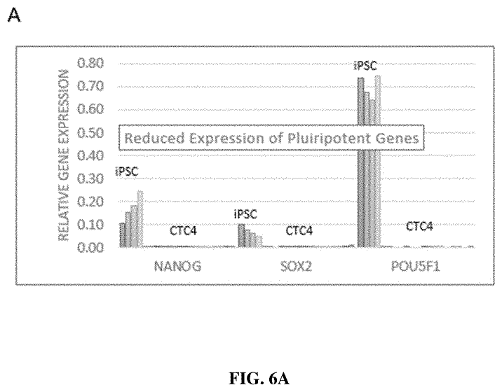

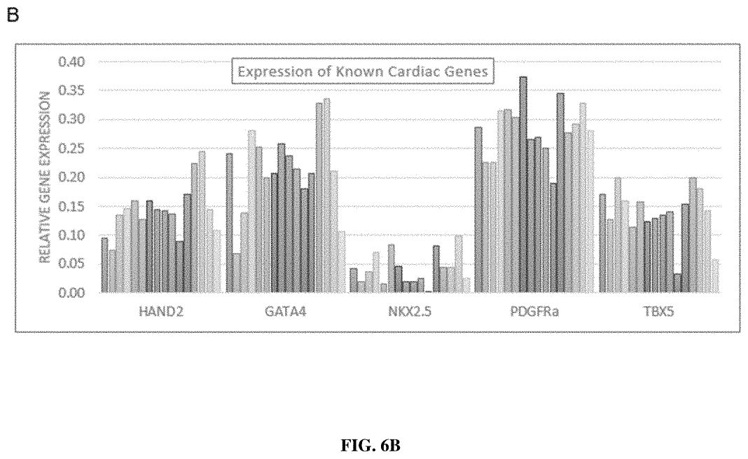

The cells obtained according to the present methods can be characterized according to a number of phenotypic criteria. CTC4 cells can have down regulated pluripotent genes while expressing known cardiac genes shown in FIGS. 6A-B and characterized by a unique cell surface marker combination of CD56⁺PDGFRA⁺KDR⁻CXCR4⁻EPCAM⁻ (FIG. 7B). Cardiomyocytes and precursor cells derived from pluripotent stem cell lines often have morphological characteristics of cardiomyocytes from other sources. They can be spindle, round, triangular or multi-angular shaped, and they may show striations characteristic of sarcomeric structures detectable by immunostaining. They may form flattened sheets of cells, or aggregates that stay attached to the substrate or float in suspension, showing typical sarcomeres and atrial granules when examined by electron microscopy.

Pluripotent stem cell-derived cardiomyocytes and their precursors typically have at least one of the cardiomyocyte specific markers including cardiac troponin I (cTnI), a subunit of troponin complex that provides a calcium-sensitive molecular switch for the regulation of striated muscle contraction, cardiac troponin T (cTnT), or Nkx2.5, a cardiac transcription factor expressed in cardiac mesoderm during early mouse embryonic development, which persists in the developing heart. The cells will also typically express at least one (and often at least 3, 5, or more) of the markers including Atrial natriuretic factor (ANF), myosin heavy chain (MHC), particularly the β chain which is cardiac specific, MLC, Titin, tropomyosin, α-sarcomeric actinin, and desmin. ANF is a hormone expressed in developing heart and fetal cardiomyocytes but down-regulated in adults. It is considered a good marker for cardiomyocytes because it is expressed in a highly specific manner in cardiac cells but not skeletal myocytes. Additional markers include MEF-2A, MEF-2B, MEF-2C, MEF-2D (transcription factors that are expressed in cardiac mesoderm and persist in developing heart), N-cadherin, which mediates adhesion among cardiac cells, Connexin 43, which forms the gap junction between cardiomyocytes, β1-adrenoceptor (β1-AR), creatine kinase MB (CK-MB) and myoglobin, which are elevated in serum following myocardial infarction, α-cardiac actin, early growth response-I, cyclin D2, and GATA-4, a transcription factor that is highly expressed in cardiac mesoderm and persists in the developing heart. It regulates many cardiac genes and plays a role in cardiogenesis.

Tissue-specific markers can be detected using any suitable immunological technique—such as flow immunocytometry or affinity adsorption for cell-surface markers, immunocytochemistry (for example, of fixed cells or tissue sections) for intracellular or cell-surface markers, Western blot analysis of cellular extracts, and enzyme-linked immunoassay, for cellular extracts or products secreted into the medium. Antibodies that distinguish cardiac markers like cTnI and cTnT from other isoforms are available commercially from suppliers like Sigma and Spectral Diagnostics. Expression of an antigen by a cell is said to be antibody-detectable if a significantly detectable amount of antibody will bind to the antigen in a standard immunocytochemistry or flow cytometry assay, optionally after fixation of the cells, and optionally using a labeled secondary antibody.

The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods using publicly available sequence data (GenBank). Expression of tissue-specific markers as detected at the protein or mRNA level is considered positive if the level is at least or about 2-, 3-, 4-, 5-, 6-, 7-, 8-, or 9-fold, and more particularly more than 10-, 20-, 30, 40-, or 50-fold above that of a control cell, such as an undifferentiated pluripotent stem cell or other unrelated cell type.

Once markers have been identified on the surface of cells of the desired phenotype, they can be used for immunoselection to further enrich the population by techniques such as immunopanning or antibody-mediated fluorescence-activated cell sorting.

Under appropriate circumstances, pluripotent stem cell-derived cardiomyocytes often show spontaneous periodic contractile activity. This means that when they are cultured in a suitable tissue culture environment with an appropriate Ca²⁺ concentration and electrolyte balance, the cells can be observed to contract across one axis of the cell, and then release from contraction, without having to add any additional components to the culture medium. The contractions are periodic, which means that they repeat on a regular or irregular basis, at a frequency between about 6 and 200 contractions per minute, and often between about 20 and about 90 contractions per minute in normal buffer. Individual cells may show spontaneous periodic contractile activity on their own, or they may show spontaneous periodic contractile activity in concert with neighboring cells in a tissue, cell aggregate, or cultured cell mass.

The contractile activity of the cells can be characterized according to the influence of culture conditions on the nature and frequency of contractions. Compounds that reduce available Ca²⁺ concentration or otherwise interfere with transmembrane transport of Ca²⁺ often affect contractile activity. For example, the L-type calcium channel blocker diltiazem inhibits contractile activity in a dose-dependent manner. On the other hand, adrenoceptor agonists like isoprenaline and phenylephrine have a positive chronotropic effect. Further characterization of functional properties of the cell can involve characterizing channels for Na⁺, K⁺, and Ca²⁺. Electrophysiology can be studied by patch clamp analysis for cardiomyocyte like action potentials. See Igelmund et al., 1999; Wobus et al., 1995; and Doevendans et al., 2000.

Functional attributes provide a manner of characterizing cells and their precursors in vitro, but may not be necessary for some of the uses referred to in this disclosure. For example, a mixed cell population enriched for cells bearing some of the markers listed above, but not all of the functional or electrophysiology properties, can be of considerable therapeutic benefit if they are capable of grafting to impaired cardiac tissue, and acquiring in vivo the functional properties needed to supplement cardiac function.

Where derived from an established line of pluripotent stem cells, the cell populations and isolated cells of the present disclosure can be characterized as having the same genome as the line from which they are derived. This means that the chromosomal DNA will be over 90% identical between the pluripotent stem cells and the cardiac cells, which can be inferred if the cardiac cells are obtained from the undifferentiated line through the course of normal mitotic division. The characteristic that cardiomyocyte lineage cells are derived from the parent cell population is important in several respects. In particular, the undifferentiated cell population can be used for producing additional cells with a shared genome—either a further batch of cardiac cells, or another cell type that may be useful in therapy—such as a population that can pre-tolerize the patient to the histocompatibility type of the cardiac allograft (US 2002/0086005; WO 03/050251).

### IV. METHODS OF USE

The CTC4 cells or cells derived therefrom, such as cardiomyocytes, vascular endothelial cells, or smooth muscle cells, provided by methods and compositions of certain aspects can be used in a variety of applications. These include but are not limited to transplantation or implantation of the cells in vivo; screening cytotoxic compounds, carcinogens, mutagens growth/regulatory factors, pharmaceutical compounds, etc., in vitro; elucidating the mechanism of cardiac diseases and injuries; studying the mechanism by which drugs and/or growth factors operate; diagnosing and monitoring cancer in a patient; gene therapy; and the production of biologically active products.

CTC4 cells or cells derived therefrom, such as cardiomyocytes, vascular endothelial cells, or smooth muscle cells, of the present disclosure can be used commercially to screen for factors (such as solvents, small molecule drugs, peptides, oligonucleotides) or environmental conditions (such as culture conditions or manipulation) that affect the characteristics of such cells and their various progeny.

In some aspects, CTC4 cells or cells derived therefrom, such as cardiomyocytes, vascular endothelial cells, or smooth muscle cells, are used to screen factors that promote maturation into later-stage cardiomyocyte precursors, or terminally differentiated cells, or to promote proliferation and maintenance of such cells in long-term culture. For example, candidate maturation factors or growth factors are tested by adding them to cells in different wells, and then determining any phenotypic change that results, according to desirable criteria for further culture and use of the cells.

Other screening applications of the present disclosure relate to the testing of pharmaceutical compounds for their effect on cardiac muscle tissue maintenance or repair. Screening may be done either because the compound is designed to have a pharmacological effect on the cells, or because a compound designed to have effects elsewhere may have unintended side effects on cells of this tissue type. The screening can be conducted using any of the precursor cells or terminally differentiated cells of the disclosure.

The reader is referred generally to the standard textbook In vitro Methods in Pharmaceutical Research, Academic Press, 1997, and U.S. Pat. No. 5,030,015. Assessment of the activity of candidate pharmaceutical compounds generally involves combining the differentiated cells of this disclosure with the candidate compound, either alone or in combination with other drugs. The investigator determines any change in the morphology, marker phenotype, or functional activity of the cells that is attributable to the compound (compared with untreated cells or cells treated with an inert compound), and then correlates the effect of the compound with the observed change.

Cytotoxicity can be determined in the first instance by the effect on cell viability, survival, morphology, and the expression of certain markers and receptors. Effects of a drug on chromosomal DNA can be determined by measuring DNA synthesis or repair. [³H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to Vickers (pp 375-410 in In vitro Methods in Pharmaceutical Research, Academic Press, 1997) for further elaboration.

Effect of cell function can be assessed using any standard assay to observe phenotype or activity of cardiomyocytes, such as marker expression, receptor binding, contractile activity, or electrophysiology—either in cell culture or in vivo. Pharmaceutical candidates can also be tested for their effect on contractile activity—such as whether they increase or decrease the extent or frequency of contraction. Where an effect is observed, the concentration of the compound can be titrated to determine the median effective dose (ED₅₀).

The present disclosure further provides methods for screening for agents that have an effect on human cardiovascular progenitor cells, cardiovascular colonies, cardiomyocytes, endothelial cells and vascular smooth muscle cells. The method comprises contacting cells from one of the cell populations described hereinabove with a candidate agent, and determining whether the agent has an effect on the cell population. The agent to be tested may be natural or synthetic, one compound or a mixture, a small molecule or polymer including polypeptides, polysaccharides, polynucleotides and the like, an antibody or fragment thereof, a compound from a library of natural or synthetic compounds, a compound obtained from rational drug design, a condition such as a cell culture condition, or any agent the effect of which on the cell population may be assessed using assays known in the art. The effect on the cell population may be determined by any standard assay for phenotype or activity, including for example an assay for marker expression, receptor binding, contractile activity, electrophysiology, cell viability, survival, morphology, or DNA synthesis or repair. Standard proliferation and differentiation assays are described in U.S. Pat. No. 6,110,739. Such agents are useful for the control of cell growth, differentiation and survival in vivo and in vitro, and tissue maintenance, regeneration and repair.

### A. Pharmaceutical Compositions

The present disclosure further provides compositions comprising populations of committed cardiac progenitor cells or cells derived therefrom, such as cardiomyocytes, vascular endothelial cells, or smooth muscle cells. The compositions may comprise pharmaceutically acceptable carriers and diluents. The compositions may further comprise components that facilitate engraftment. Compositions comprising these populations are useful for cell and tissue replacement and repair, and for generating populations of cardiomyocytes in vitro and in vivo. Compositions comprising CTC4 cells are useful for expansion of the progenitor populations. The compositions may be formulated as a medicament or delivery device for treating a cardiac condition.

The CTC4 cells or cells derived therefrom, such as cardiomyocytes, vascular endothelial cells, or smooth muscle cells, of the present disclosure can be supplied in the form of a pharmaceutical composition, comprising an isotonic excipient prepared under sufficiently sterile conditions for human administration. In certain aspects, it may be desirable to disperse the cells using a protease or by gentle mechanical manipulation into a suspension of single cells or smaller clusters. To reduce the risk of cell death upon engraftment, the cells may be treated by heat shock or cultured with about 0.5 U/mL erythropoietin about 24 hours before administration.

For general principles in medicinal formulation, the reader is referred to Cell Therapy: Stem Cell Transplantation, Gene Therapy, and Cellular Immunotherapy, 1996; and Hematopoetic Stem Cell Therapy, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cardiomyocytes. Suitable ingredients include matrix proteins that support or promote adhesion of the cardiomyocytes, or complementary cell types, especially endothelial cells.

This disclosure also includes a reagent system, comprising a set or combination of cells that exist at any time during manufacture, distribution, or use. The cell sets comprise any combination of two or more cell populations described in this disclosure, exemplified but not limited to a type of differentiated cell (cardiomyocytes, cardiomyocyte precursors, and so on), in combination with undifferentiated pluripotent stem cells or other differentiated cell types, often sharing the same genome. Each cell type in the set may be packaged together, or in separate containers in the same facility, or at different locations, at the same or different times, under control of the same entity or different entities sharing a business relationship.

Pharmaceutical compositions of this disclosure may optionally be packaged in a suitable container with written instructions for a desired purpose, such as the reconstitution of CTC4 cells or cells derived therefrom, such as cardiomyocytes, vascular endothelial cells, or smooth muscle cells, to improve a disease condition or abnormality of the cardiac muscle.

### B. Therapeutic Uses

The cells provided in certain aspects of this present disclosure can be used for therapy of any subject in need thereof. Human conditions that may be appropriate for such therapy include cardiac disorders, such as myocardial infarction, cardiomyopathy, congestive heart failure, ventricular septal defect, atrial septal defect, congenital heart defect, ventricular aneurysm, a cardiac disorder which is pediatric in origin, ventricular aneurysm, or a cardiac disorder which requires ventricular reconstruction.

For human therapy, the dose is generally between about 10⁸ and 10¹² cells, and typically between about 2×10⁸ and 1×10⁹ cells, making adjustments for the body weight of the subject, nature and severity of the affliction, and the replicative capacity of the administered cells. The ultimate responsibility for determining the mode of treatment and the appropriate dose lies with the managing clinician.

Certain aspects also provide for the use of CTC4 cells to enhance tissue maintenance or repair of cardiac muscle for any perceived need, such as an inborn error in metabolic function, the effect of a disease condition, or the result of significant trauma.

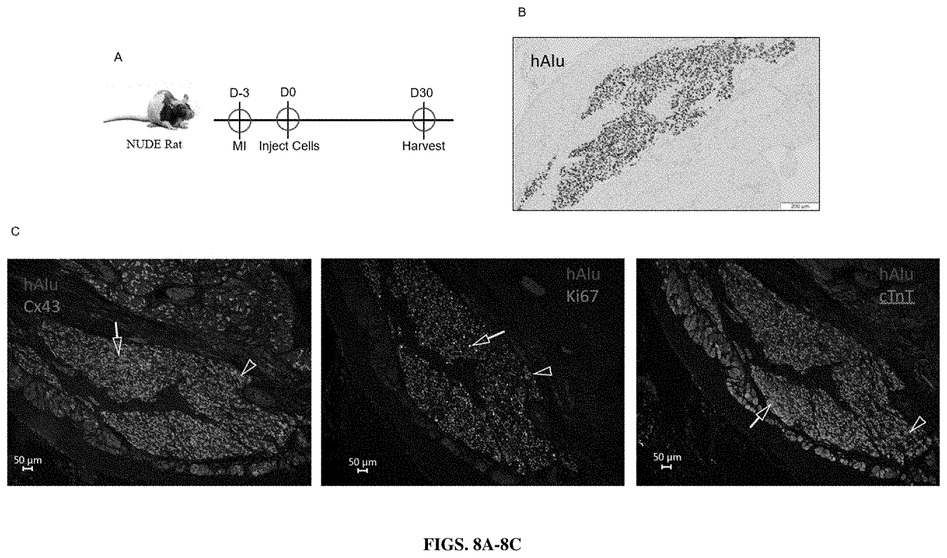

To determine the suitability of cell compositions for therapeutic administration, the cells can first be tested in a suitable animal model. At one level, cells are assessed for their ability to survive and maintain their phenotype in vivo. Cell compositions are administered to immunodeficient animals (such as NUDE rats, or animals rendered immunodeficient chemically or by irradiation). Tissues are harvested after a period of engraftment, and assessed as to whether pluripotent stem cell-derived cells are still present. CTC4 cells were shown to engraft and survive at least 30 days post injection (FIG. 8B). The CTC4 cells can also continue to differentiate to cardiomyocytes as shown in FIG. 8C by co-staining the hAlu⁺ cells with the gap junction protein connexin 43 (CX43) and the structural protein cardiac troponin T (CTNT).

Other methods to track cells in vivo may be by administering cells that express a detectable label (such as green fluorescent protein, or β-galactosidase); that have been prelabeled (for example, with BrdU or [³H]thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

Suitability can also be determined by assessing the degree of cardiac recuperation that ensues from treatment with a cell population of cardiomyocytes derived from pluripotent stem cells. A number of animal models are available for such testing. For example, hearts can be cryoinjured by placing a precooled aluminum rod in contact with the surface of the anterior left ventricle wall (Murry et al., 1996; Reinecke et al., 1999; U.S. Pat. No. 6,099,832; Reinecke et al., 2004). In larger animals, cryoinjury can be effected by placing a 30-50 mm copper disk probe cooled in liquid N₂ on the anterior wall of the left ventricle for about 20 min (Chiu et al., 1995). Infarction can be induced by ligating the left main coronary artery (Li et al., 1997). Injured sites are treated with cell preparations of this disclosure, and the heart tissue is examined by histology for the presence of the cells in the damaged area. Cardiac function can be monitored by determining such parameters as left ventricular end-diastolic pressure, developed pressure, rate of pressure rise, and rate of pressure decay.

After adequate testing, differentiated cells of this disclosure can be used for tissue reconstitution or regeneration in a human patient or other subject in need of such treatment. The cells are administered in a manner that permits them to graft or migrate to the intended tissue site and reconstitute or regenerate the functionally deficient area. Special devices are available that are adapted for administering cells capable of reconstituting cardiac function directly to the chambers of the heart, the pericardium, or the interior of the cardiac muscle at the desired location.

Where desirable, the patient receiving an allograft of pluripotent stem cell-derived CTC4 cells can be treated to reduce immune rejection of the transplanted cells. Methods contemplated include the administration of traditional immunosuppressive drugs like cyclosporin A (Dunn et al., Drugs 61:1957, 2001), or inducing immunotolerance using a matched population of pluripotent stem cell-derived cells (WO 02/44343; U.S. Pat. No. 6,280,718; WO 03/050251). Another approach is to adapt the CTC4 cell population to decrease the amount of uric acid produced by the cells upon transplantation into a subject, for example, by treating them with allopurinol. Alternatively or in conjunction, the patient is prepared by administering allopurinol, or an enzyme that metabolizes uric acid, such as urate oxidase (PCT/US04/42917).

Patients suitable for receiving regenerative medicine according to the present methods include those having acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

In another embodiment, the present disclosure provides methods of cell replacement and methods of tissue replacement useful for treatment of disorders characterized by insufficient cardiac function including, for example, congenital heart disease, coronary heart disease, cardiomyopathy, endocarditis and congestive heart failure. Both the differentiated cells and the cardiovascular progenitor cells are useful for replacement therapy, since the progenitor populations are capable of differentiation to the cardiomyocyte, endothelial and vascular smooth muscle lineages in vivo. The cells are also useful for generating cardiovascular tissue in vitro. Methods for engineering cardiac tissue are known in the art and reviewed for example by Birla in “Stem Cell Therapy and Tissue Engineering for Cardiovascular Repair” Springer, 2006. Accordingly, in one embodiment the present disclosure provides a method of cardiomyocyte replacement therapy comprising administering to a subject in need of such treatment a composition comprising cardiomyocytes isolated from a cell population enriched for human cardiovascular progenitor cells obtained in accordance with the present disclosure. In another embodiment, the present disclosure provides a method of treating a disorder characterized by insufficient cardiac function comprising administering to a subject in need of such treatment a composition comprising human cardiovascular progenitor cells. In a preferred embodiment, the subject is a human. The composition may be administered by a route that results in delivery to or migration to cardiac tissue including, for example, injection or implantation, and under conditions that result in a reduction of at least one adverse effect or symptom or the disorder.

With respect to the therapeutic methods of the present disclosure, it is not intended that the administration of CTC4 cells to a mammal be limited to a particular mode of administration, dosage, or frequency of dosing; the present disclosure contemplates all modes of administration, including intramuscular, intravenous, intrarticular, intralesional, subcutaneous, or any other route sufficient to provide a dose adequate to prevent or treat a disease. The CTC4 cells may be administered to the mammal in a single dose or multiple doses. When multiple doses are administered, the doses may be separated from one another by, for example, one week, one month, one year, or ten years. One or more growth factors, hormones, interleukins, cytokines, small molecules or other cells may also be administered before, during, or after administration of the cells to further bias them towards a particular cell type.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—iPSC-derived Cardiac Progenitor Cells

iPSCs were thawed and expanded on a vitronectin-coated plate (2.5 μg/mL) in Essential 8 Medium (E8) for 3 days on a feeder-free, monolayer culture. Media was exchanged daily.

On day 0 of the suspension differentiation aggregate formation and mesoderm induction was initiated by harvesting the iPSCs with TrypLE, washing with E8 and resuspending in aggregate formation medium comprising E8, 1 uM H1152 (Rho kinase inhibitor), and 2 uM CHIR99021 (Wnt agonist). The cell density was adjusted to 1×10⁶ cells/mL and bioreactors (PBS500 or PBS3) were seeded.

On day 1 the aggregates were transitioned to a new medium by allowing aggregates to settle before exchanging 80% of the medium with RPMI+B27 (without insulin) and 5 μM CHIR 99021. Day 2 aggregates were fed by allowing aggregates to settle first before exchanging 80% of the medium with RPMI, B27 (without insulin), and 4.4 μM CHIR 99021.

For cardiac specification, day 3 aggregates were first allowed to settle before exchanging 80% of the medium with RPMI, B27 (with insulin) and 10 uM XAV939 (Wnt inhibitor). In some instances, for specific iPSCs, additional small molecules were added to efficiently induce cardiac specification. For example, these small molecules included 2 uM SB431542 (TGFβ/Activin inhibitor) and/or 1-2 uM Dorsomorphin (BMP inhibitor).

As the cells continued to specify towards the cardiomyocyte lineage, the cultures were fed similar to previous days. On days 4 and 5, the aggregates were settled first before exchanging 80% of the medium with RPMI and B27 (with insulin). The entire process was serum-free and no drug resistant selection was used.

On day 6, the cells had a committed fate, but had not yet differentiated to cardiomyocytes. The aggregates were harvested and washed with D-PBS (-/-) before dissociating with TrypLE and cryopreserving as a single cell suspension in CryoStor CS10 using a controlled rate freezer.

The cardiac progenitor cells were analyzed for cardiac mesoderm markers (i.e., KDR, CKIT, and PDFGRα) and cardiomyocytes markers (i.e., SAA and SMA). The differentiation method resulted in cardiomyocytes with over 95% SAA. Thus, the present methods efficiently produced committed cardiac progenitor cells and cardiomyocytes.

### Example 2—Identifying Differentiation Stage to Add Wnt Inhibition

The time point during cardiac differentiation that requires Wnt inhibition has never been well described. The cell surface markers CXCR4 and CD56 can be used to monitor the state of the cultures and help determine when Wnt inhibition should be used.

Aggregate samples at days 1-6 were taken from both PBS500 or PBS3 bioreactor cultures, dissociated, and stained for CXCR4 and CD56. Each day the cell populations shifted to different expression profiles, first showing the expression of CXCR4 followed by double positive CXCR4^{pos}CD56^{pos} cells and finally losing the expression of CXCR4 by days 4-6.

If the cultures expressed too much CD56 on day 3 or had already begun to lose expression of CXCR4, it was too late to inhibit Wnt signaling and specify the cultures towards a cardiomyocyte fate. However, it was found that efficient cardiac specification occurred if the CXCR4 positive populations just began to express CD56 as well. Robust expression of CD56 was one indication that either too much CHIR 99021 was used or the cell densities were too low.

Another indication of potential cardiac differentiation failure was if too much CXCR4 was expressed on day 2. This was a clear indication that too much CHIR 99021 was used previous to day 2.

### Example 3—Differentiation Scale

This cardiac differentiation was first developed using the PBS500 vessels, however, this scale was too small to manufacture cell therapy doses of 1×10⁸-1×10⁹ cells (FIG. 2B). The volumes, PBS wheel speed, pH, and dissolved oxygen were examined using the PBS500 format and applied to optimize a clinical relevant differentiation scale using multiple PBS3 bioreactors.

### Example 4—Cryopreservation Scale

Standard 1.5-2.0 ml cryovials can be used to cryopreserve small scale samples of iPSC-derived products. Multiple different size cryovials were tested from Aseptic Technologies with the goal of cryopreserving enough committed cardiac progenitor cells in one vial that can be used as a clinical dose. Multiple AT vial sizes were tested, and it was determined that the AT6 vials allowed for a clinically relevant dose per vial (FIG. 2C). Freezing up to 300×10⁶ cells per vial was tested with the resulting cells passing all of the quality release assays.

### Example 4—Committed Cardiac Progenitor Cells Express Specific Markers

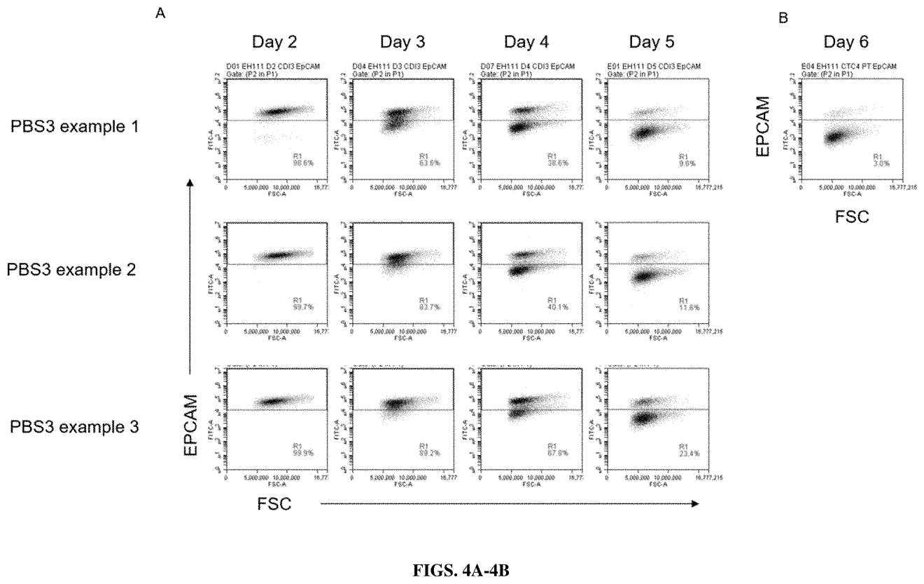

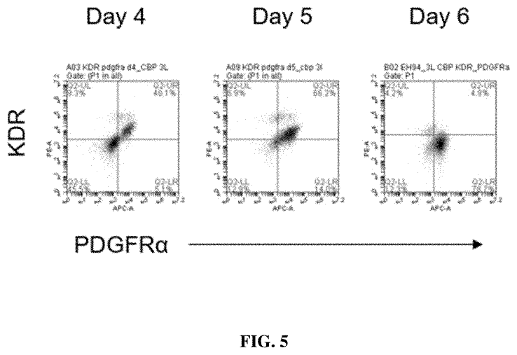

A time course study was performed that tested multiple markers each day during the differentiation process. There was a clear induction from day 1 to day 3 of CXCR4 and PDGFRα indicating the specification of cardiac mesoderm (FIG. 3B). The dynamic expression of KDR was also detected where the highest expression was seen on day 4 followed by the rapid down regulation during days 5 and 6 (FIG. 5A). CD56 was also induced during day 3 and was maintained throughout the differentiation process. Expression of EpCAM was monitored to decrease more each day during the process and resulted in less than 10% positive cells by day 6 (FIG. 4). Cardiomyocyte structural proteins were also minimally expressed by day 6.

### Example 5—Committed Cardiac Progenitor Cells Become Cardiomyocytes

The day 6 CTC4 cells were thawed and plated to test their cardiomyocyte differentiation potential. Different densities were seeded onto vitronectin-coated vessels in RPMI and B27 (with insulin) and cultured for around 7 days (FIG. 7A). Medium was changed every other day with a full volume change. The monolayers began contracting 2-6 days after being plated. The contracting cells were harvested and analyzed by flow cytometry for cardiomyocyte specific markers. The cells analyzed were more than 90% sarcomeric alpha actinin positive (FIG. 7D).

The day 6 CTC4 cells were also plated into vitronectin-coated 96 well plates in RPMI and B27 (with insulin) and cultured for 7 days. The cells were stained by immunocytochemistry for various cardiomyocyte markers and stained positive for cardiac troponin T, cardiac troponin I, and sarcomeric alpha actinin. The cells also stained for the cardiac-specific transcription factor NKX2.5 (FIG. 7C).

### Example 6—Committed Cardiac Progenitor Cells (CTC4) Engraft in Myocardial Infarction Model

A NUDE rat myocardial infarct model was used to test the engraftment and differentiation of the CTC4 cells (FIGS. 8A-C). Three days after infarction, CTC4 cells were thawed, counted, and resuspended in 5% Flexbumin. Cells were administered by direct injection into multiple injection sites. One month after injection the hearts were harvested and stained for human cells using immunohistochemistry or in situ hybridization detection methods for human Alu. Once human cells were found in specific sites within the rat myocardium additional serial sections were processed and stained for cardiac troponin T (cardiomyocyte), Ki67 (proliferation), and CX43 (gap junction) markers using immunohistochemistry methods. Human cells were detected one month post injection and robustly expressed cardiac troponin T and CX43 indicating the CTC4 cells continued to differentiate in vivo and became electrically coupled cardiomyocytes. In addition, a small amount of cells were also stained for Ki67 indicating the potential for the human graft sites to sightly expand.

### Example 7—Committed Cardiac Progenitor Cell Differentiation to Vascular Endothelial Cells or Smooth Muscle Cells

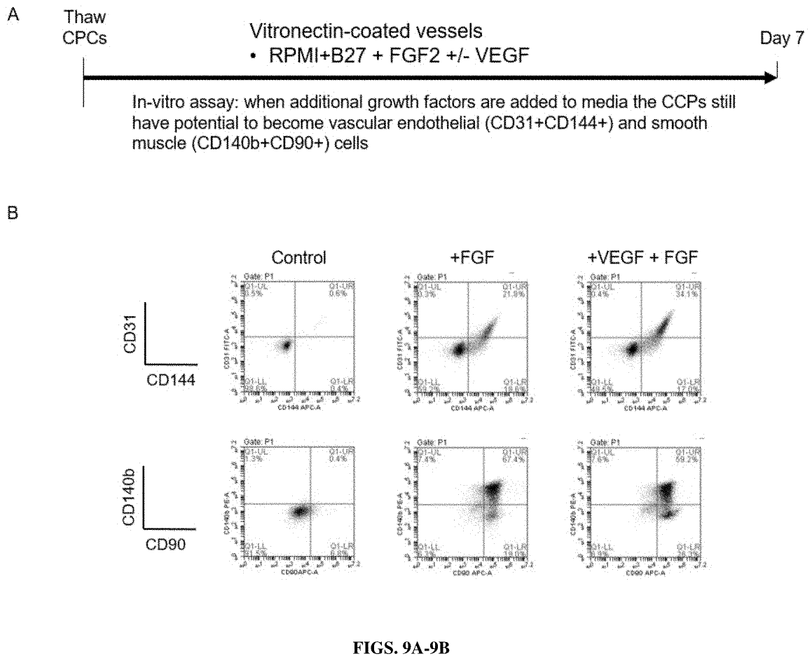

Studies were performed to show that the committed cardiac progenitor cells have the potential to further differentiate to other cell lineages, such as endothelial cells (CD31+CD144+) and smooth muscle cells (CD140b+CD90+) (FIG. 9). The iPSC-derived committed cardiac progenitor cells were cultured in RPMI+B27 media containing specific growth factors. The committed cardiac progenitor cells were cultured for about 7 days in media comprising FGF and/or VEGF to produce vascular endothelial cells or smooth muscle cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Amit et al., Dev. Bio., 227:271-278, 2000.

### Byrne et al., Nature, 450 (7169):497-502, 2007.

Doevendans et al., J. Mol. Cell Cardiol., 32:839, 2000.

### Dunn et al., Drugs 61:1957, 2001.

### Fernandes, et al., J. Biotechnology, 132 (2):227-236, 2007.

### Igelmund et al., Pflugers Arch., 437:669, 1999.

International Patent Publication No. PCT/JP2009/062911
International Patent Publication No. PCT/JP2011/069588
International Patent Publication No. WO 02/44343
International Patent Publication No. WO 03/050251
International Patent Publication No. WO 03/050251
International Patent Publication No. WO 2007/069666
International Patent Publication No. WO 2007/069666
International Patent Publication No. WO2005/123902
International Patent Publication No. WO98/30679
Keller et al., Curr. Opin. Cell Biol., 7:862-869, 1995.

### Klimanskaya et al., Lancet., 365:P1636-1641, 2005.

Ludwig et al., Nat. Biotechnol., 24:185-187, 2006b.
Ludwig et al., Nat. Methods, 3:637-646, 2006a.

### Takahashi et al., Cell, 131, 861-872, 2007.

Thomson and Marshall, Curr. Top. Dev. Biol., 38:133-165, 1998.

### Thomson and Odorico, Trends Biotechnol., 18 (2):53-57, 2000.

Thomson et al. Proc. Natl. Acad. Scie. USA, 92:7844-7848, 1995.
U.S. application Ser. No. 12/478,154
U.S. Pat. No. 8,546,140
U.S. Pat. No. 5,843,780
U.S. Pat. No. 5,843,780
U.S. Pat. No. 6,103,470
U.S. Pat. No. 6,110,739
U.S. Pat. No. 6,200,806
U.S. Pat. No. 6,280,718
U.S. Pat. No. 6,416,998
U.S. Pat. No. 7,029,913
U.S. Pat. No. 7,442,548
U.S. Pat. No. 7,598,364
U.S. Pat. No. 7,989,425
U.S. Pat. No. 8,058,065
U.S. Pat. No. 8,071,369
U.S. Pat. No. 8,129,187
U.S. Pat. No. 8,183,038
U.S. Pat. No. 8,268,620
U.S. Pat. No. 8,268,620
U.S. Pat. No. 8,546,140
U.S. Pat. No. 8,546,140
U.S. Pat. No. 8,691,574
U.S. Pat. No. 8,691,574
U.S. Pat. No. 8,741,648
U.S. Pat. No. 8,741,648
U.S. Pat. No. 8,741,648
U.S. Pat. No. 8,900,871
U.S. Pat. No. 9,175,268

### U.S. Patent Publication No. 2003/0211603

### U.S. Patent Publication No. 2009/0246875

### U.S. Patent Publication No. 2010/0210014

### U.S. Patent Publication No. 2011/0104125

### U.S. Patent Publication No. 2012/0276636

### U.S. Patent Publication No. 2015/0191697

U.S. Patent Publication No. US 2002/0086005

### United States Patent 20070116680

### Watanabe et al., Nature Neurosci., 8:288-296, 2005.

Wobus et al., Ann. N.Y. Acad. Sci., 27:752, 1995.
Xu et al., Nat. Biotechnol., 19:971-974, 2001.

### Yu et al., Science, 318: 1917-1920, 2007.

1. An in vitro method for producing human pluripotent stem cell (PSC)-derived committed cardiac progenitor cells comprising:
(a) culturing PSCs in the presence of a Wnt agonist to initiate differentiation and a survival agent to form cell aggregates;
(b) further culturing the cell aggregates in the presence of a Wnt agonist for a period of time sufficient to produce a population of mesoderm cells; and
(c) differentiating the population of mesoderm cells in the presence of a Wnt inhibitor to promote cardiac specification, thereby producing a population of committed cardiac progenitor cells.

2. The method of claim 1, wherein the PSCs are induced pluripotent stem cells (iPSCs) or embryonic stem cells (ESCs).

3-4. (Canceled)

5. The method of claim 1, wherein the survival agent is a Rho-associated kinase (ROCK) inhibitor or blebbistatin.

6. (canceled)

7. (canceled)

8. The method of claim 1, wherein the method comprises culturing cells in suspension culture in one or more bioreactors.

9. (canceled)

10. The method of claim 1, wherein the Wnt agonist of step (a) is CHIR 99021, SB216763, CHIR 98014, TWS119, Tideglusib, SB415286, BIO, AZD2858, AZD1080, AR-A014418, TDZD-8, LY2090314, or IM-12.

11-14. (canceled)

15. The method of claim 1, wherein the Wnt signaling agonist of step (b) is CHIR 99021, SB216763, CHIR 98014, TWS119, Tideglusib, SB415286, BIO, AZD2858, AZD1080, AR-A014418, TDZD-8, LY2090314, or IM-12.

16-18. (canceled)

19. The method of claim 1, wherein the culture of step (b) further comprises an Activin/Nodal agonist and/or BMP.

20-22. (canceled)

23. The method of claim 1, wherein at least 5% of the population of mesoderm cells express CD56 prior to or during step (c) and/or wherein at least 40% of the population of mesoderm cells express KDR and PDGFRα prior to or during step (c).

24-26. (canceled)

27. The method of claim 1, wherein step (c) comprises adding a Wnt inhibitor when at least 30% positive of the population of mesoderm cells are positive for CXCR4 and less than 60% of the population of mesoderm cells are positive for CD56.

28. (canceled)

29. The method of claim 1, wherein the Wnt inhibitor of step (c) is XAV939, IWR1, IWR2, IWR3, IWR4, ICG-001, IWR-1-endo, Wnt-059, LGK-974, LF3, CP21R7, NCB-0846, PNU-74654, or KYA179K.

30. (canceled)

31. (canceled)

32. The method of claim 1, wherein the culture of step (c) further comprises a TGFβ inhibitor.

33. The method of claim 32, wherein the TGFβ inhibitor is SB431542, LDN-193189, LY2157299, LY2109761, SB525334, SIS HCl, SB505124, GW788388, or LY364947.

34-36. (canceled)

37. The method of claim 1, wherein the culture of step (c) further comprises a BMP inhibitor or AMPK inhibitor.

38. The method of claim 37, wherein the BMP inhibitor is dorsomorphin, LDN193189, DMH1, DMH2, or ML 347.

39-47. (canceled)

48. The method of claim 1, wherein less than 20% of the population of committed cardiac progenitor cells are positive for KDR, EpCAM, CXCR4, and/or SAA.

49. (canceled)

50. The method of claim 1, wherein at least 80% of the population of committed cardiac progenitor cells are positive for PDGFRα and CD56.

51. (canceled)

52. The method of claim 1, further comprising cryopreserving the population of committed cardiac progenitor cells that are at least 70% positive for PDGFRα, less than 40% positive for KDR, less than 20% positive for EpCAM, and less than 20% for SAA.

53. (canceled)

54. The method of claim 1, further comprising maturing the population of committed cardiac progenitor cells to produce cardiomyocytes that express CTNT, MHC, MLC, CTNI, and/or sarcomeric alpha actinin, wherein the culture for maturation does not comprise a Wnt inhibitor or a TGFβ inhibitor.

55-62. (canceled)

63. The method of claim 1, further comprising differentiating the population of committed cardiac progenitor cells to a population of vascular endothelial cells or smooth muscle cells in the presence of fibroblast growth factor (FGF) and/or vascular endothelial growth factor (VEGF).

64-71. (canceled)

72. A composition comprising a population of committed cardiac progenitor cells with at least 90% expression of CD56, 80% positive for PDGFRα and less than 10% expression of CXCR4, KDR and EpCAM.

73-75. (canceled)

76. A method for the treatment of a cardiac disorder in a subject comprising administering an effective amount of committed cardiac progenitor cells produced by the method of claim 1 to a subject in need thereof.

77-84. (canceled)

85. A method of generating cardiac progenitor cells, comprising:
(a) providing pluripotent stem cells (PSCs);
(b) culturing the PSCs in suspension in the presence of a Wnt agonist to initiate cardiac differentiation; and
(c) adding a Wnt inhibitor when the cell population is comprised of less than 60% CD56-positive cells and more than 30% CXCR4-positive cells to promote robust cardiac specification, thereby producing a population of cardiac progenitor cells.

86-99. (canceled)
